(19) United States
(12) Patent Application Publication
(10) Pub. No.: US 2025/0295523 A1
(43) Pub. Date: Sep. 25, 2025

(54) SYSTEMS AND METHODS FOR DELIVERING A RETINAL PATCH

(71) Applicant: Alcon Inc., Fribourg (CH)

(72) Inventors: Stephen Paul Blizzard, Mission Viejo, CA (US); Jiaxuan Li, Rancho Cucamonga, CA (US); Matthew Douglas McCawley, Grant, FL (US); Quinton A. Quintana, Corona, CA (US)

(21) Appl. No.: 19/084,075

(22) Filed: Mar. 19, 2025

Related U.S. Application Data
(60) Provisional application No. 63/567,541, filed on Mar. 20, 2024.
(60) Provisional application No. 63/695,454, filed on Sep. 17, 2024.

(51) Int. Cl. A61F 9/00; A61M 5/19; A61M 5/20

(52) U.S. Cl. CPC A61F 9/0017; A61M 5/19; A61M 5/2046; A61M 2202/064; A61M 2205/7545

(57) ABSTRACT

A mixer attachment for use with a surgical hand piece includes a powder plate configured to contain a solid substance for combining with a liquid substance to form a combined substance. The mixer attachment further includes a plurality of mixing plates configured to mix the solid substance and the liquid substance in the combined substance, the plurality of mixing plates including: one or more channeled plates comprising one or more channels and one or more through holes configured to facilitate turbulent flow of the combined substance during mixing, and one or more flat plates comprising a central opening to direct flow of the combined substance between the one or more channeled plates.

This application claims the benefits under 35 USC § 119(e) of U.S. provisional application Nos. 63/567,541 filed on 20 Mar. 2024, and 63/695,454 filed on 17 Sep. 2024, herein incorporated by reference in their entireties.

### INTRODUCTION

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed.

In certain ophthalmic surgical procedures in the posterior segment of the eye, such as vitrectomies for treating retinal detachments, a retinal break may need to be blocked and the retina held in place in order to prevent fluid from flowing through the break and facilitate healing of the retina following surgery. Current methods of blocking retinal breaks and holding the retina in place include infusing silicone oil, an expansile gas, or other surgical tamponade into the eye after a vitrectomy procedure in order to hold the retina in place and allow it to heal.

However, the use of a surgical tamponade in retinal detachment management may require a follow-up procedure in addition to the initial treatment, and/or may cause unwanted effects in a patient's eye. For example, when silicone oil is used as a tamponade in the treatment of retinal detachment, a second surgery is needed to remove the silicone oil from the eye. In certain cases, the silicone oil may also cause the development of cataracts in the eye. Alternatively, the utilization of an expansile gas as a tamponade does not require a second surgery to remove the expansile gas, but the patient may be required to position their head at a particular angle for many hours, days, or weeks, such that the gas bubble is positioned over a retinal hole or tear.

Retinal patches have recently been developed as a replacement for expansile gas and/or silicone oil tamponades. They provide improved surgical efficiency and outcomes, and fewer postoperative restrictions for patients. However, in order to deliver a retinal patch, a surgeon typically has to mix material constituents (e.g., a liquid and powder) to form a retinal patch mixture prior to delivery thereof to a target site. The surgeon must also monitor the amount of time between the mixing of materials and the application of the retinal patch to the patient's eye to avoid premature curing of the retinal patch. This creates difficulty and inefficiency in the procedure, and unpredictability of the retinal patch mixture.

Therefore, improved methods and procedures for delivery of retinal patches, as well as other tamponades, for treating retinal detachment are desirable.

### SUMMARY

Aspects of the present disclosure relate to a surgical device for mixing and delivering a substance in ophthalmic procedures, such as hand pieces for mixing and delivering retina patch to the eye for use in ophthalmic surgical procedures.

In some embodiments, a mixer attachment for use with a surgical hand piece is provided, the mixer attachment comprising: a powder plate, the powder plate configured to include a solid substance for combining with a liquid substance to form a combined substance; and a plurality of mixing plates configured to mix the solid substance and liquid substance in the combined substance, the plurality of mixing plates comprising: one or more channeled plates, the one or more channeled plates each comprising one or more channels and one or more through holes configured to facilitate diverging and converging flow of the combined substance during mixing; and one or more flat plates, the one or more flat plates each comprising an opening to direct flow of the combined substance between the one or more channeled plates.

In some embodiments, a surgical hand piece is provided, the surgical hand piece comprising: a syringe, the syringe comprising a connector at a distal end of the syringe; a plunger, the plunger coupled to a drive system and configured to translate toward the distal end of the syringe upon actuation of the plunger; and a mixer attachment removably coupled to the connector at the distal end of the syringe.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates a perspective view of an exemplary tethered hand piece for use in ophthalmic surgery, according to embodiments described herein.

FIG. 1B illustrates a block diagram of a surgical console in connection with the hand piece of FIG. 1A, according to some embodiments herein.

FIG. 2 illustrates a perspective cross-sectional view of the tethered hand piece of FIG. 1A, according to embodiments described herein.

FIG. 3 illustrates a perspective view of an exemplary untethered hand piece for use in ophthalmic surgery, according to embodiments described herein.

FIG. 4A-4B illustrate side cross-sectional views of the untethered hand piece of FIG. 3 in a relaxed or an active position, respectively, according to embodiments described herein.

FIGS. 4C-4D illustrate side views of the untethered hand piece of FIG. 3, without a housing, in a relaxed or an active position, respectively, according to embodiments described herein.

FIGS. 4E-4F illustrate enlarged side cross-sectional views of a portion of the untethered hand piece of FIG. 3, in a relaxed or an active position, respectively, according to embodiments described herein.

FIG. 5A illustrates a perspective view of an exemplary untethered hand piece for use in ophthalmic surgery, according to embodiments described herein.

FIG. 5B illustrates a perspective cross-sectional view of the untethered hand piece of FIG. 5A, according to embodiments described herein.

FIG. 5C illustrates a side cross-sectional view of the untethered hand piece of FIG. 5A, without a housing, according to embodiments described herein.

FIG. 5D illustrates a side view of the untethered hand piece of FIG. 5A, without a housing, according to embodiments described herein.

FIG. 6A illustrates a cross-sectional view of an exemplary mixer attachment, according to embodiments described herein.

FIG. 6B illustrates an exploded view of the mixer attachment of FIG. 6A, according to embodiments described herein.

FIGS. 7A and 7B illustrate front views of exemplary channeled plates of a mixer attachment, according to embodiments described herein.

FIG. 8 illustrates a front view of an exemplary flat plate of a mixer attachment, according to embodiments described herein.

FIG. 9A illustrates a perspective view of a powder plate of the mixer attachment of FIG. 6A, according to embodiments described herein.

FIG. 9B illustrates a side cross-sectional view of the powder plate of FIG. 9A, according to embodiments described herein.

FIG. 9C illustrates a perspective view of the powder plate of FIG. 9A, in addition to components configured to be supported within the powder plate during use, according to embodiments described herein.

FIG. 10A illustrates a perspective view of an exemplary variable dose stop mechanism, according to embodiments described herein.

FIG. 10B illustrates an exploded view of the variable dose stop mechanism of FIG. 10A, according to embodiments described herein.

FIGS. 11A-11C illustrate a perspective cross-sectional view, a perspective phantom view, or a perspective view, respectively, of a lock ring of the variable dose stop mechanism of FIG. 10A, respectively, according to embodiments described herein.

FIGS. 12A-12C illustrate a perspective cross-sectional view, a perspective phantom view, or a perspective view, respectively, of a dose ring of the variable dose stop mechanism of FIG. 10A, respectively, according to embodiments described herein.

FIGS. 13A-13D illustrate various positions of the variable dose stop mechanism of FIGS. 10A, according to embodiments herein.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Aspects of the present disclosure relate to a surgical device for mixing and delivering a substance during ophthalmic (eye) procedures, such as, a mixing attachment for a surgical instrument for use in ophthalmic surgical procedures. The systems and methods described herein provide improved surgical efficiency, improved surgical outcomes, improved mixing predictability, and fewer postoperative restrictions for patients.

Examples will now be described relative to the Drawings.

Note that, as described herein, a “distal” end, side, or portion of a component refers to the end, side, or the portion that is further from the user's body and closer to the patient's body during the use thereof. On the other hand, a “proximal” end, side, or portion of the component refers to the end, side, or portion that is distanced closer to the user's body and further away from the patient's body.

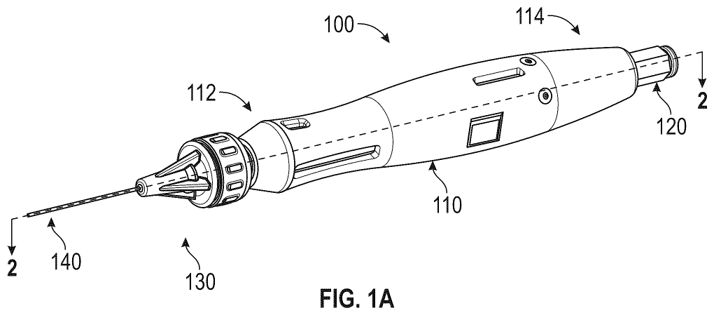

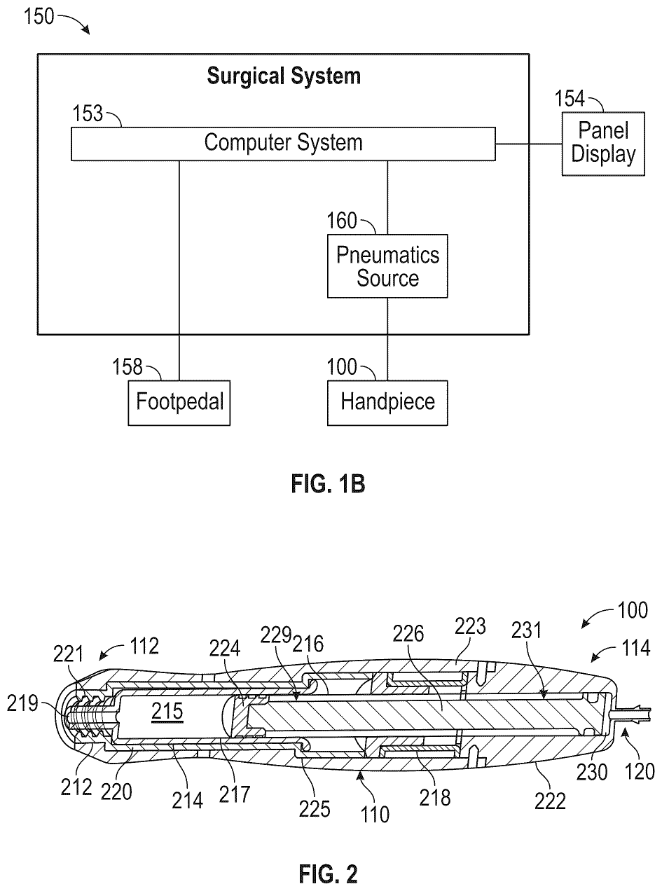

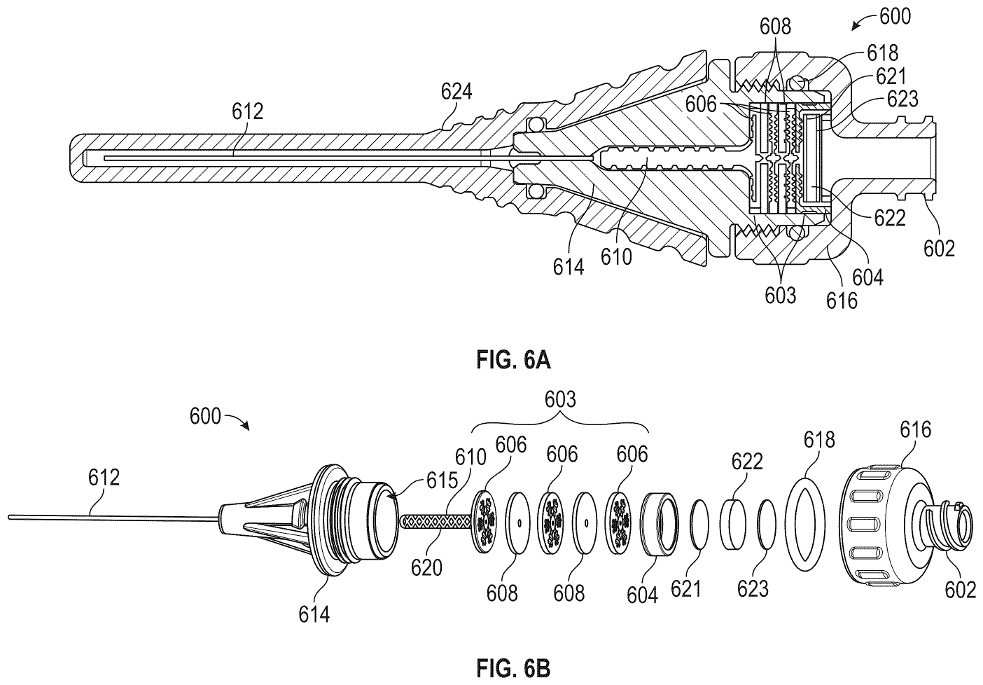

FIG. 1A illustrates a perspective view of a tethered hand piece 100 and a mixer attachment 130 for use in ophthalmic procedures, including ophthalmic surgical procedures like retinal detachment procedures, according to embodiments described herein. In some embodiments, the hand piece 100 is an injector hand piece configured to be utilized in conjunction with the mixer attachment 130, which is described in more detail with reference to FIGS. 6A-9C (as mixer attachment 600). As depicted in FIG. 1A, the hand piece 100 includes an outer housing 110 having a distal end 112 configured to be coupled with the mixer attachment 130, and a proximal end 114 having a connection port 120. The hand piece 100 may be coupled, or tethered, to a surgical console via the connection port 120. In some embodiments, the connection port 120 of the hand piece 100 is configured to receive, over the connection port 120, a flexible tube coupled to a corresponding port on the surgical console, fluidically coupling the hand piece 100 to the surgical console.

The surgical console may include, or be in communication with, a hand piece drive system operably coupled to the hand piece 100 (e.g., via the one or more flexible tubes attached to the connection port 120) for driving the dispensing of fluid substances from the hand piece 100. In some embodiments, the hand piece drive system includes a pneumatic drive system having a pressurized gas source configured to provide pressurized gas to the hand piece 100. In some embodiments, the pressurized gas is used to translate a plunger or other similar device in the hand piece 100 towards the distal end 112 of the outer housing 110. The movement of the plunger, in turn, forces fluid substance(s), found in a syringe and/or cartridge of the hand piece 100, out of the hand piece 100, through the mixer attachment 130, and out of a cannula 140 of the mixer attachment 130. Thus, during an ophthalmic surgical procedure, the cannula 140 of the mixer attachment 130, when coupled to the hand piece 100, may be inserted into an eye of a patient to deliver a substance to a target site within the patient's eye, such as a retinal break, in response to the provision of, e.g., pneumatic pressure or other driving force, from the surgical console to the hand piece 100. In some embodiments, the hand piece drive system is controlled via manual or automatic inputs (e.g., depression of the foot pedal of the surgical system 150 in FIG. 1B, for example).

FIG. 1B illustrates a block diagram of a surgical console in operable connection with the hand piece 100, according to some embodiments. The surgical system 150 (e.g., a surgical console) includes a computer system 153 and an associated display screen 154 showing data related to system operation and performance during an ophthalmic surgical procedure. The surgical system 150 further includes at least part of a number of components which are used together to perform an ophthalmic surgical procedure. For example, the surgical system 150 includes a user input device (e.g., a foot pedal 158) and a hand piece drive system (e.g., a pneumatics source 160) in communication with the hand piece 100. These components may overlap and cooperate to perform various aspects of the procedure. For example, in some embodiments, manual actuation of the foot pedal 158 by a surgeon may provide an input to the computer system 153, which is then used to activate the pneumatics source 160 and drive operation of the hand piece 100 to dispense fluid.

Computer system 153 controls operation of the surgical system 150. Generally, computer system 153 includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C, C++, Java, Python, Rust, or any other programming language (including assembly languages, hardware description languages, and database programming languages). In some instances, the code may be a program that, when loaded into the processor, causes the surgical system 150 to receive and process information from one or more of foot pedal 158 and pneumatics source 160, for, e.g., providing fluid control for one or more hand pieces 100 or other devices in communication with the surgical system 150.

The processor may be, or include, a microprocessor, a microcontroller, an embedded microcontroller, a programmable digital signal processor, or any other programmable device operable to receive information from the memory or other devices in communication with the processor, computer system 153, and/or surgical system 150, and perform one or more operations on the received information. For example, the processor may send instructions to components of pneumatics source 160, or other devices or systems in communication with computer system 153, for controlling such devices and systems. For example, the processor may send instructions to the pneumatics source 160 to dispense fluid to the hand piece 100 for mixing of one or more components and injecting a mixed substance into the patient's eye, as described herein. The processor may also be operable to output results based on the operations performed thereby. In some embodiments, computer system 153 includes a controller that sends instructions to components of surgical system 150. A display screen 154 shows data provided by computer system 153. In some embodiments, the display screen 154 is a touch screen configured to receive input from a user for controlling components of pneumatics source 160, or other devices or systems in communication with computer system 153.

FIG. 2 illustrates a perspective cross-sectional view of the exemplary tethered hand piece 100 of FIG. 1A to better illustrate internal components thereof, according to embodiments described herein. The cross-sectional view of hand piece 100 depicts the outer housing 110, a syringe 214, a syringe sleeve 220, a plunger 216, a dose stop mechanism 218, and the connection port 120.

The syringe 214 is disposed within the syringe sleeve 220 inside a primary chamber 225 of the outer housing 110. In some embodiments, the syringe sleeve 220 is configured to interact with the dose stop mechanism 218 to facilitate the provision of two or more measured doses, e.g., as described in further detail below. As shown, both the syringe 214 and syringe sleeve 220 extend through an opening 212 at the distal end 112 of the outer housing 110, which allows for removable coupling of the syringe 214 with the mixer attachment 130 (of FIG. 1) within the opening 212. In some embodiments, the syringe sleeve 220 and/or syringe 214 include a connector 221, such as a threaded and/or Luer-type connector, disposed within the opening 212 that provides a connection point for coupling with the mixer attachment 130. In some embodiments, the connector 221 includes a snap-fit, a pressed fit, or a friction fit type connector. In some embodiments, the connector 221 is configured to mate with a corresponding connector of the mixer attachment 130 (of FIG. 1) to create a leak-free seal therebetween.

The syringe 214 is operably coupled to the plunger 216 within the primary chamber 225. The plunger 216 includes a sealing portion 224 coupled to a distal end 229 of a rod portion 226. The sealing portion 224 friction fits with an interior surface of a barrel 217 of the syringe 214 and seals off a proximal end of a volume 215 within the barrel 217 of syringe 214. In some embodiments, the volume 215 is configured to contain, or hold, a first fluid substance to be dispensed out of a discharge orifice 219 at a distal end of the syringe 214 upon distal movement of the plunger 216, which can then be mixed with a second fluid or solid substance to form, e.g., a retinal patch.

A proximal end 231 of the rod portion 226 of the plunger 216 is in fluidic communication with the connection port 120 via a rod chamber 230 at the proximal end 114 of the outer housing 110. In some embodiments, the rod chamber 230 is disposed through an endcap 222 of the outer housing 110 disposed at the proximal end 114. During use, upon activation of a hand piece drive system in communication with the hand piece 100 (e.g., pneumatics source 160), a fluid or gas may be flowed through the connection port 120 and into the rod chamber 230. As the rod chamber 230 fills with fluid, the plunger 216 is translated toward the distal end 112 of the outer housing 110, causing the first fluid substance to be expelled from the discharge orifice 219 of the syringe 214 disposed through the opening 212 in the outer housing 110. In some embodiments, the first fluid substance may comprise a polyethylene glycol or other viscous liquid.

In some embodiments, the syringe sleeve 220, syringe 214, dose stop mechanism 218, and/or plunger 216 are removable from within the outer housing 110, such that the syringe sleeve 220, syringe 214, dose stop mechanism 218, and/or plunger 216 can be exchanged between uses. For example, in some embodiments, the endcap 222 may be removable from and/or hingedly attached to a main body 223 of the outer housing 110, which facilitates removal and/or exchange of the syringe 214, syringe sleeve 220, and/or dose stop mechanism 218 from the outer housing 110. In such embodiments, the outer housing 110, including the main body 223 and the endcap 222, may be autoclavable or sterilizable.

Note that while described as a “syringe” and “plunger,” the syringe 214 and plunger 216 may include any suitable type of containment device and piston, respectively, for containing and dispensing fluids, such as ophthalmic fluid substances or other surgical substances.

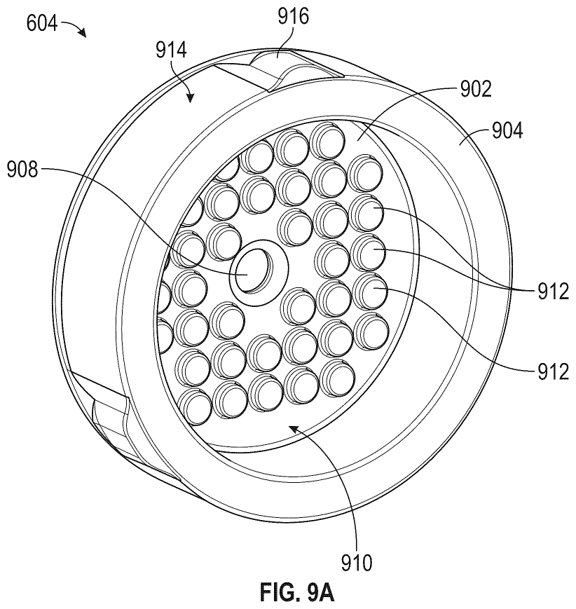

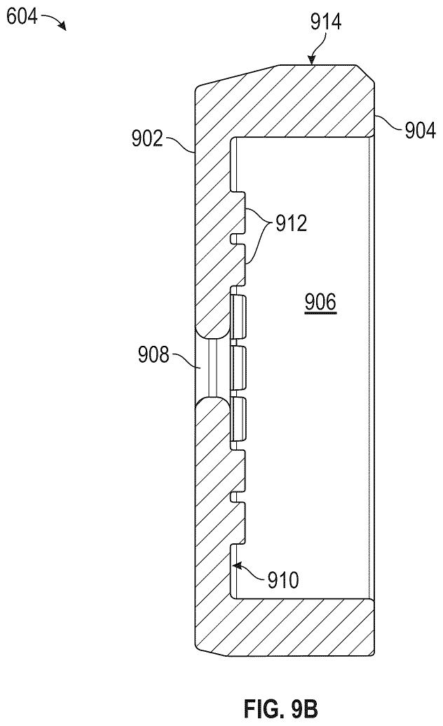

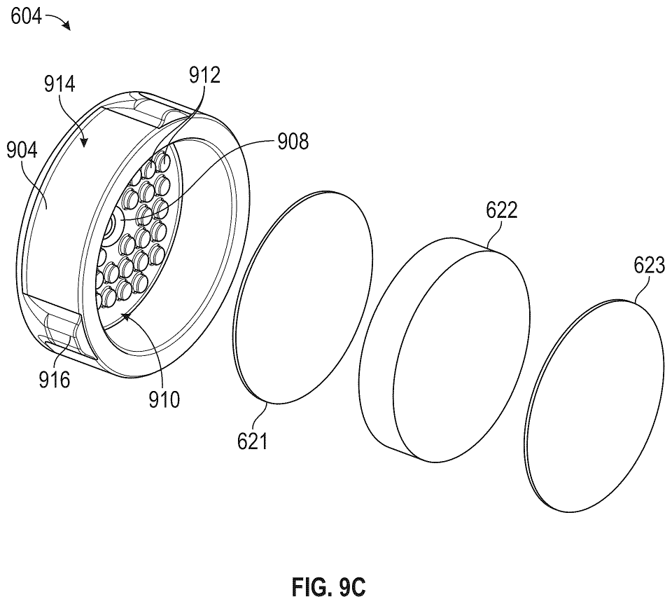

In some embodiments, the dose stop mechanism 218 may allow the plunger 216 to translate only a predetermined distance towards the distal end of the hand piece 100 with each drive input received from the surgical console to facilitate provision of a target dose volume, e.g., as discussed in more detail in reference to FIGS. 8-9.

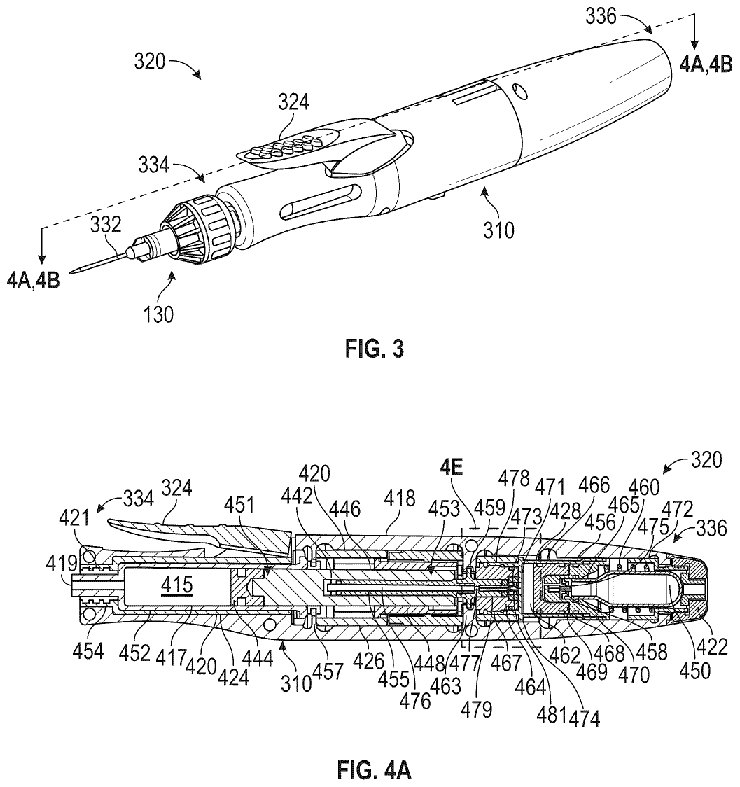

FIG. 3 illustrates a perspective view of an exemplary untethered hand piece 320 for use in ophthalmic surgery, including ophthalmic surgical procedures like retinal detachment procedures, according to embodiments described herein. Similar to the tethered hand piece 100, in some embodiments, the untethered hand piece 320 is an injector hand piece configured to be utilized in conjunction with the mixer attachment 130 (of FIG. 1), which is described in more detail with reference to FIGS. 6A-9C (as mixer attachment 600).

As depicted in FIG. 3, the surgical hand piece 320 includes an outer housing 310 having a distal end 334 configured to be coupled with the mixer attachment 130, a lever 324, and a proximal end 336. The surgical hand piece 320 may be a separate instrument that is not physically coupled, or tethered, to the surgical console (e.g., unlike the surgical hand piece described with reference to FIG. 1). As such, a connection port may not be needed or used at the proximal end 336.

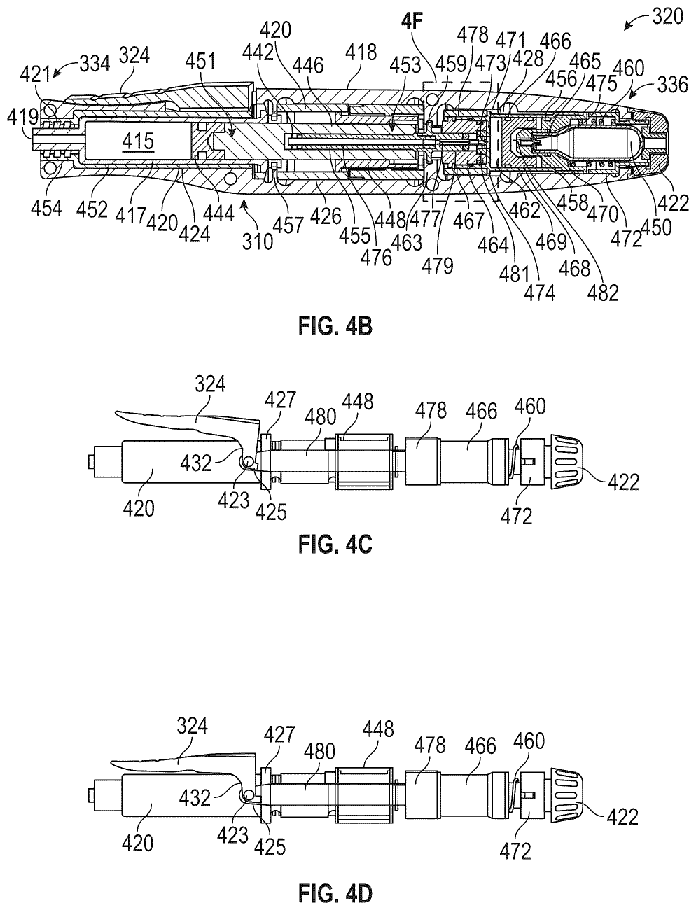

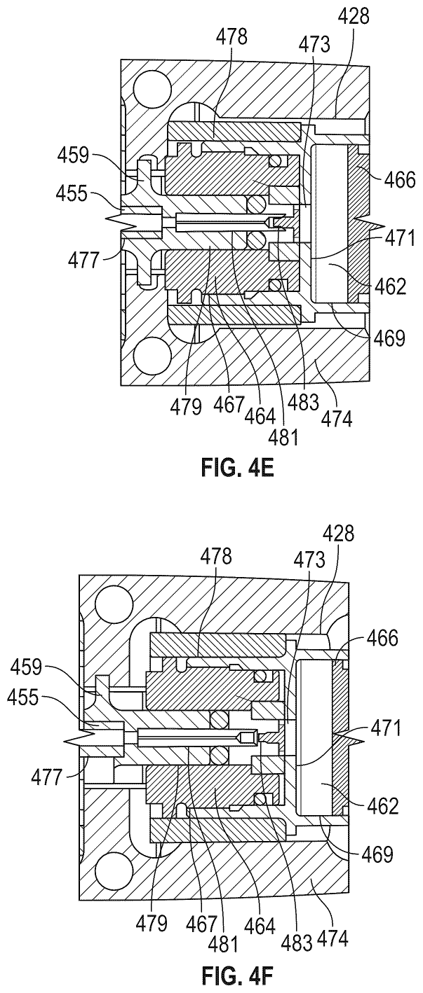

The lever 324 acts as a user input device to facilitate user control of the dispensing of fluid substances from the hand piece 320. Though the lever 324 is shown in FIG. 3, a different user input device may also be utilized, such as a button, a slider, a wheel, a deformable basket, or other similar toggle. In some embodiments, a surgeon or other healthcare professional may manually depress the lever 324, or actuate the other similar toggle, to cause dispensing of fluid substances from the hand piece 320. In some embodiments, the lever 324 or other toggle is operably coupled to and configured to cause actuation of an internal drive system within the hand piece 320, which may drive fluid substance(s), found in a syringe or cartridge within the hand piece 320, out of the surgical hand piece 320, through the mixer attachment 130, and out of a cannula 332 of the mixer attachment 130. The cannula 332 may be placed in a patient's eye in order to allow delivery of the fluid substance through the cannula 332 and into the eye. The fluid may include a retina patch useful as a replacement for expansile gas and/or silicone oil tamponades to support the retina for healing. The retina patch may also be used, for example, to seal a tear or hole in the retina (e.g., seal an injection site after injecting a drug sub-retinally. Other uses for retina patches are also contemplated. The drive system of the hand piece 320 is discussed in more detail with reference to FIGS. 4A-4F.

FIGS. 4A-4B illustrate cross-sectional views of the hand piece 320 in a relaxed or an active position, respectively, to better illustrate the internal drive system thereof, according to embodiments described herein. FIGS. 4C-4D illustrate side views of the hand piece 320, with the outer housing 310 removed to show various internal components, in both the relaxed or the active position, respectively, according to some embodiments described herein. FIGS. 4E-4F illustrate enlarged side cross-sectional views of a portion of the hand piece 320, in a relaxed or an active position, according to some embodiments described herein.

Turning to FIGS. 4A-4B, the hand piece 320 includes the outer housing 310, the lever 324 movably coupled to and extending from the outer housing 310, a syringe 452, a syringe sleeve 420, a plunger 442 including a sealing portion 444 and a rod portion 446, a dose stop mechanism 448, and an internal, pre-charged pneumatic drive system including a gas canister 450 and a piston assembly 456, in addition to other components as discussed in more detail below. In the relaxed position (FIGS. 4A and 4C), the lever 324 is undepressed or in a non-actuated state; conversely, in the active position (FIGS. 4B and 4D), the lever 324 is depressed.

The syringe 452 is disposed within the syringe sleeve 420 inside a primary chamber 424 of the outer housing 310 located nearest the distal end 334 of the outer housing 310. In some embodiments, the syringe sleeve 420 is configured to interact with the dose stop mechanism 448 to facilitate the provision of two or more measured doses, which is described in further detail below. As shown, both the syringe 452 and syringe sleeve 420 extend through an opening 454 at the distal end 334, which allows for coupling of the syringe 452 with the mixer attachment 130 (of FIG. 1) within the opening 454. In some embodiments, the syringe sleeve 420 and/or syringe 452 include a threaded connector 421, such as a Luer-type connector, disposed within the opening 454 that provides a connection point for coupling with the mixer attachment 130 (of FIG. 1). In some embodiments, the threaded connector 421 is configured to mate with a corresponding threaded connector of the mixer attachment 130 to create a leak-free seal therebetween.

The syringe 452 is operably coupled to the plunger 442 within the primary chamber 424. The plunger 442 includes the sealing portion 444 coupled to a distal end 451 of the rod portion 446. The sealing portion 444 friction fits with an interior surface of a barrel 417 of the syringe 452 and seals off a proximal end of a volume 415 within the barrel 417 of syringe 452. During use, the volume 415 is configured to contain a first fluid substance, which can be dispensed out of a discharge orifice 419 at the distal end of the syringe 452 upon distal movement of the plunger 442, which can then mix with a second fluid or solid substance to form, e.g., a retinal patch. In some embodiments, the fluid substance may comprise a polyethylene glycol or other viscous liquid.

In some embodiments, the syringe sleeve 420, syringe 452, dose stop mechanism 448, and/or plunger 442 are removable from within the outer housing 310, such that the syringe sleeve 420, syringe 452, dose stop mechanism 448, and/or plunger 442 can be exchanged between uses. In such embodiments, the outer housing 310 may be autoclavable or sterilizable.

Note that while described as a “syringe” and “plunger,” the syringe 452 and plunger 442 may include any suitable type of containment device and piston, respectively, for containing and dispensing fluid substances, such as ophthalmic fluid substances and other surgical substances.

A proximal end 453 of the rod portion 446 is disposed through the dose stop mechanism 448 within a secondary chamber 426 of the outer housing 310. The proximal end 453 is further slidably coupled with an intermediary conduit 455 that extends distally into the secondary chamber 426. In some embodiments, the intermediary conduit 455 includes a hollow tubular structure having a lumen 476 in fluidic communication with a drive chamber 428 (e.g., a tertiary chamber) of the hand piece 320. In the example shown, the proximal end 453 of the rod portion 446 slides over the intermediary conduit 455 such that the intermediary conduit 455 is disposed through the rod portion 446 and is circumferentially surrounded by the rod portion 446. However, in other embodiments, the proximal end 453 of the rod portion 446 may extend into, or be disposed through, the lumen 476 of the intermediary conduit 455. Together with one or more projections 457 extending from an inner wall of the secondary chamber 426, the dose stop mechanism 448 and the intermediary conduit 455 support the rod portion 446 within the secondary chamber 426 and facilitate axial alignment/orientation thereof.

A proximal end of the intermediary conduit 455 is inserted into and supported by a flanged connector 459, which is disposed between the secondary chamber 426 and the drive chamber 428 adjacent to the proximal end 336 of the outer housing 310. In the illustrated examples, the intermediary conduit 455 extends into a central channel 477 of the flanged connector 459; however, in other embodiments, the flanged connector 459 extends into the lumen 476 of the intermediary conduit 455. The flanged connector 459 acts as a fluidic junction between the various components in the drive chamber 428 and the intermediary conduit 455 and rod portion 446 in the secondary chamber 426. In some embodiments, a flange of the flanged connector 459 is mated with a slot of a projection 463 extending inwardly from the outer housing 310. The projection 463 may circumferentially surround the flanged connector 459 and support the flanged connector 459 between the secondary chamber 426 and the drive chamber 428. In some embodiments, the projection 463 partially defines each of the secondary chamber 426 and the drive chamber 428.

A proximal end of the flanged connector 459 extends into the drive chamber 428 and slidably mates with a connector 464 of the piston assembly 456. In the illustrated examples, the flanged connector 459 slidably extends into a central channel 479 of the connector 464; however, in other embodiments, the connector 464 has a portion that slidably extends into the central channel 477 of the flanged connector. The piston assembly 456 further includes a piston sleeve 466, a piston 468, and a puncture pin attachment 470 coupled to a puncture pin 458.

The piston sleeve 466 is generally cylindrical in shape and is moveable within the drive chamber 428. A flange 474 extends radially outward from an outer surface of the piston sleeve 466 and is configured to engage with a collar 478 mechanically coupled to the lever 324 to facilitate actuation of the piston assembly 456. In some embodiments, the piston sleeve 466 includes a first cavity 467 and a second cavity 469, which are separated by a circular partition 471 having a central opening 473 disposed therein for fluidically coupling the first cavity 467 and the second cavity 469. As shown in FIGS. 4A-4B, the connector 464 is disposed and secured within the first cavity 467, while the piston 468 and puncture pin attachment 470 are at least partially disposed within and/or secured to the second cavity 469. The connector 464 may be secured within the first cavity 467 via a threaded connection or other similar mating arrangement. Similarly, the puncture pin attachment 470 may be secured within the second cavity 469 via a threaded connection or other similar mating arrangement.

The piston 468 is slidably disposed within the second cavity 469 such that the piston 468 can slide (e.g., translate) axially within the second cavity 469 relative to the puncture pin attachment 470. When the piston 468 slides distally away from the puncture pin attachment 470, a volume 482 is formed between the piston 468 and the puncture pin attachment 470. A distal end of the piston 468 interfaces with a volume 462 within the second cavity 469 of the piston sleeve 466, while a proximal end of the piston 468 interfaces with the puncture pin attachment 470. In some embodiments, the volume 462 is configured to be filled or occupied with a fluid, such as air, silicone oil, or saline solution, that is driven through the central opening 473 and through the connector 464 upon distal translation of the piston 468. Other liquids and gases are also contemplated for the volume 462. In some embodiments, the proximal end of the piston 468 comprises a concave or convex surface configured to nest with a corresponding convex or concave surface, respectively, on a distal end of the puncture pin attachment.

A proximal end of the puncture pin attachment 470 is configured to interface with the gas canister 450 disposed at the proximal end 336 of the hand piece 320. In some embodiments, the proximal end of the puncture pin attachment 470 includes one or more concave surfaces configured to receive and/or slidably couple with a nozzle 465 of the gas canister 450. The puncture pin 458 extends from the proximal end of the puncture pin attachment 470 and is configured to puncture a seal of the nozzle 465 of the gas canister 450 upon proximal movement of the piston sleeve 466 and thus, proximal movement of the puncture pin attachment 470. The puncture pin attachment 470 further includes a conduit 475 configured to direct gas released from the gas canister 450 upon puncturing toward the proximal end of the piston 468, which drives the piston 468 distally.

The gas canister 450 is disposed proximal to the piston assembly 456 within the drive chamber 428. In some embodiments, the gas canister 450 is at least partially supported within the drive chamber 428 via a gas canister sleeve 472, which is disposed circumferentially around at least a portion of the gas canister 450 to secure the gas canister 450 within the drive chamber 428. As shown in FIGS. 4A-4B, a return spring 460 is also disposed circumferentially around the gas canister 450, such that the return spring 460 is disposed radially between the gas canister 450 and a portion of the gas canister sleeve 472. A distal end of the return spring 460 is nested (e.g., pressed) against a surface on the proximal end of the puncture pin attachment 470, while a proximal end of the return spring 460 is nested against the gas canister sleeve 472 or an inner wall of the outer housing 310. The return spring 460 is configured to distally return the piston assembly 456 to a resting state after the piston assembly 456 has been proximally actuated toward the gas canister 450. In some embodiments, the return spring 460 includes a coil spring, though other types of spring are also contemplated.

In some embodiments, the gas canister 450 is removable from within the outer housing 310, such that the gas canister 450 can be exchanged between administrations of doses. For example, in some embodiments, the outer housing 310 includes an endcap 422, which may be removable from and/or hingedly attached to a main body 418 of the outer housing 310, which facilitates removal and/or exchange of the gas canister 450 and/or other internal components.

During use, a user depresses the lever 324 as shown in FIG. 4B, which is pivotally attached to a link 480 (shown in FIGS. 4C-4D and further described below) that distally extends through the outer housing 310 from the collar 478. Depression of the lever 324 causes the link 480, and the collar 478 fixedly or removably attached thereto, to slide proximally toward the proximal end 336 of the outer housing 310. Because the collar 478 engages with the flange 474 of the piston sleeve 466, the piston sleeve 466 is also translated proximally, along with the connector 464 and puncture pin attachment 470 statically coupled therewith, as well as the piston 468. Accordingly, it may be said that the entire piston assembly 456 is proximally translated upon depression of the lever 324.

The proximal movement of the puncture pin attachment 470 causes the puncture pin 458 to puncture the seal of the nozzle 465 on the gas canister 450, which releases gas from the gas canister 450 into the conduit 475 of the puncture pin attachment 470. The released gas creates a distally-acting force on the piston 468, causing the piston 468 to slide distally through the second cavity 469 and provide a corresponding distally-acting force on fluid in the volume 462. The fluid is “pressed” or “pushed” through the central channel 479 of the connector 464 and through the central channel 477 of the flanged connector 459.

As shown in FIGS. 4E and 4F, in some embodiments, the flanged connector 459 further includes an insert 481 disposed at a proximal end of the central channel 477 that restricts or controls fluid flow through the central channel 477 and other downstream components. In some embodiments, the insert 481 is a self-sealing plug that prevents fluid flow into the central channel 477 when the lever 324 is undepressed. In some embodiments, the insert 481 is sealed such that a first depression of the lever 324 is required to break the seal of the insert 481, and a second depression of the lever 324 causes flow of fluid from the volume 462 through the central channel 479.

Also shown in FIGS. 4E and 4F, in some embodiments, the connector 464 further includes a needle valve 483 disposed at a proximal end of the central channel 479. The needle valve 483 is configured to engage with the insert 481 to seal the central channel 477 when the lever 324 is undepressed and disengage from the insert 481 to open the central channel 477 when the lever 324 is depressed. The needle valve 483 can be fixedly attached to the connector 464 such that proximal translation of the piston sleeve 466 by depression of the lever 324 also causes the needle valve 483 to translate proximally. Accordingly, depression of the lever 324 causes the needle valve 483 to translate proximally (with the connector 464) and away from the insert 481, thereby opening the insert 481 and the central channel 477 to allow fluid flow from the volume 462.

Generally, once the gas canister 450 is punctured, the volume 482 (shown in FIG. 4B) between the puncture pin attachment 470 and the piston 468 is pressurized. The volume 482 remains pressurized from that point on, which pushes the piston 468 distally and, in turn, forces fluid through the central opening 473, through the insert 481, and through the central channel 477 of the flanged connector 459.

Returning to FIGS. 4A and 4B, after passing through the central channel 479 of the connector 464, the fluid is flowed through the central channel 477 of the flanged connector 459 and into the lumen 476 of the intermediary conduit 455 in the secondary chamber 426. This pressurizes the lumen 476. The fluid then acts upon the rod portion 446 of the plunger 442 that is slidably disposed over the intermediary conduit 455 to translate the plunger 442 distally through the barrel 417 of the syringe 452. The distal movement of the plunger 442 causes the first fluid substance to be pushed through the volume 415 and dispel from the discharge orifice 419 of syringe 452.

In some embodiments, the dose stop mechanism 448 allows the plunger 442 to translate only a predetermined distance towards the distal end 334 with each depression of the lever 324 to facilitate provision of a target dose volume, as discussed in more detail with reference to FIGS. 8-9.

In some embodiments, when the gas canister 450 is punctured, the return spring 460 disposed around the gas canister 450 is compressed by the puncture pin attachment 470 of the piston assembly 456, as shown in FIG. 4B. Upon release of the lever 324, the return spring 460 counters the forces of the lever 324 on the piston assembly 456 and returns the piston assembly 456 and the lever 324 to a relaxed (e.g., unactuated) position, as shown in FIG. 4A.

Turning to FIGS. 4C-4D, side views of the hand piece 320, with the outer housing 310 removed to show various internal components, in both the relaxed and the active position, respectively, are shown. As described above, the lever 324 is pivotally attached to the link 480 (and the outer housing 310) via a pin 423 disposed through an arm 432 of the lever 324. The link 480 extends from the pin 423 to the collar 478, which is engaged with the flange 474 of the piston sleeve 466. Depression of the lever 324 thus causes the link 480, the collar 478 attached to the link 480, the piston sleeve 466 engaged with the collar 478, and the remaining components of the piston assembly 456 attached to the piston sleeve 466 to slide proximally toward the proximal end 335 of the outer housing 310. Upon release of the lever 324, the return spring 460 acts upon the piston assembly 456 to slide the piston sleeve 466, the collar 478, and the link 480 distally.

In some embodiments, the lever 324 further includes a tab 425 extending proximally from the arm 432, which interacts with a lip 427 of the syringe sleeve 420. The tab 425 is configured to prevent “run-on,” or undesired dispensing, when the lever 324 is released after depression. In the relaxed position of FIG. 4C (e.g., when the lever 324 is not depressed), the tab 425 does not contact the lip 427 of the syringe sleeve 420. In the active position of FIG. 4D, (e.g., when the lever 324 is fully depressed), the tab 425 contacts the lip 427. After the lever 324 is released following manual depression of the lever 324, the tab 425 creates a gap with the lip 427 of the syringe sleeve 420, which allows the syringe sleeve 420 to move forward slightly to relieve pressure caused by fluids within the volume 415. By relieving this pressure, the tab 425 is configured to reduce additional undesired dispensed caused by compliance or compressibility of internal components of the hand piece 320.

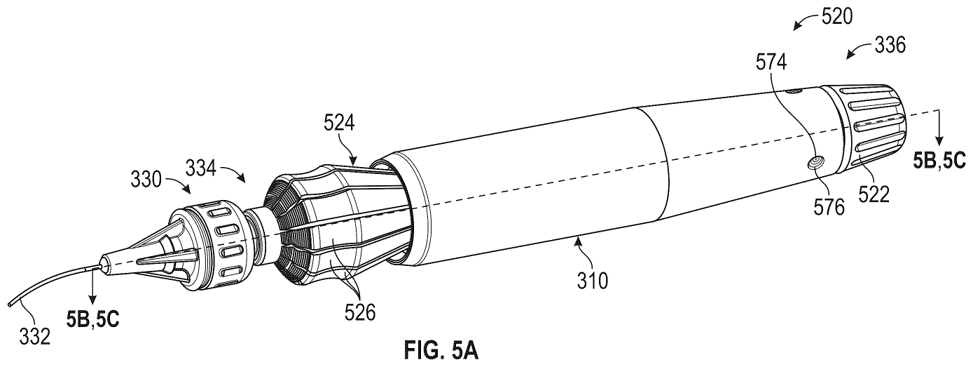

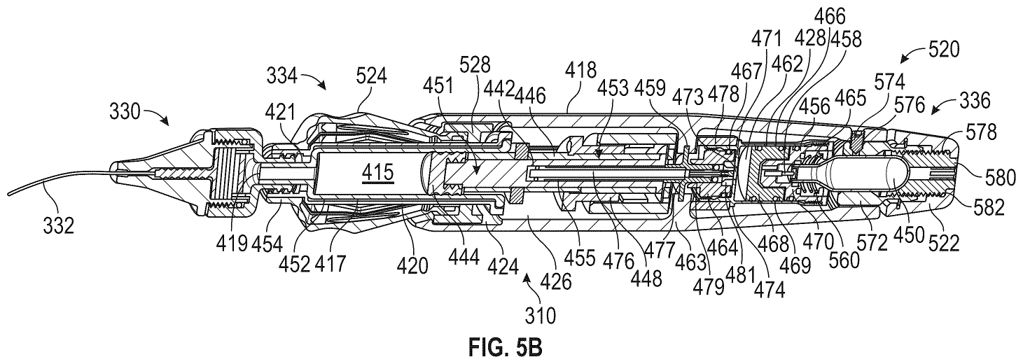

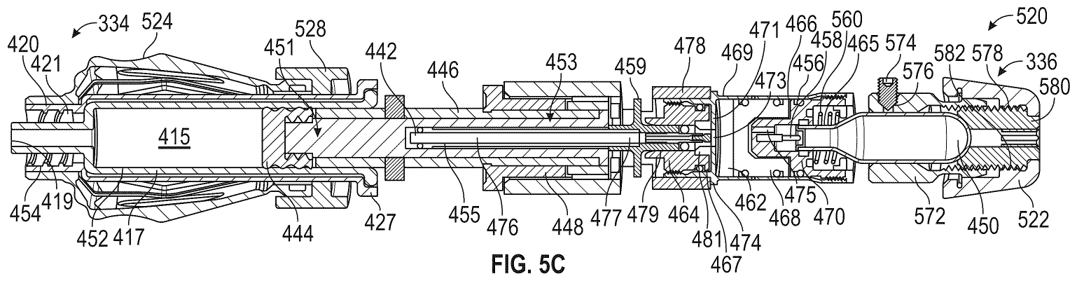

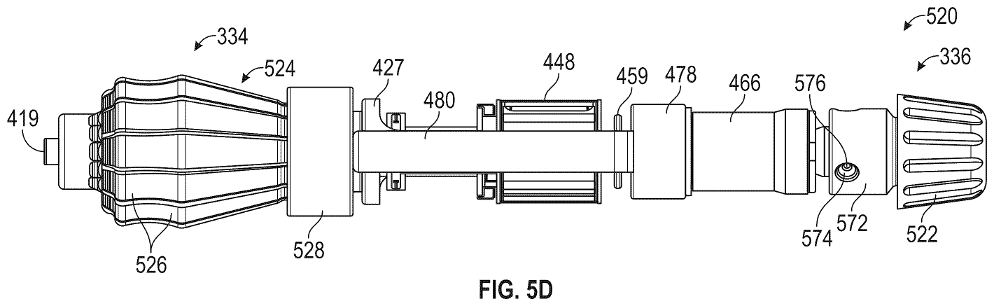

FIG. 5A illustrates a perspective view of another exemplary untethered surgical hand piece 520 for use in ophthalmic surgery, including ophthalmic surgical procedures like retinal detachment procedures, according to embodiments described herein. FIG. 5B illustrates a perspective cross-sectional view of the hand piece 520, according to embodiments described herein. FIG. 5C illustrates a side cross-sectional view of the hand piece 520, without a housing, according to embodiments described herein. And, FIG. 5D illustrates a side view of the hand piece 520, without a housing, according to embodiments described herein

The hand piece 520 is similar in arrangement and function to the hand piece 320, but includes an alternative user input device and an alternative gas cannister punctuation mechanism, which are described in more detail below. Accordingly, like components and features in FIGS. 5A-5D are indicated by identical reference numerals to FIGS. 3 and 4A-4F, and are omitted from the description below. Similar to the hand piece 320, the hand piece 520 may be configured to be utilized in conjunction with the mixer attachment 130 (of FIG. 1).

As depicted in FIG. 5A, the hand piece 520 includes a deformable basket 524 having a plurality of elastic and outwardly-curved lever arms 526 instead of a single lever. The deformable basket 524 thus acts as the user input device to facilitate user control of the dispensing of fluid substances from the hand piece 520. In some embodiments, a surgeon or other healthcare professional may manually compress the lever arms 526 of the deformable basket 524 to cause dispensing of fluid substances from the hand piece 520. The deformable basket 524 is operably coupled to and configured to cause actuation of the drive system within the hand piece 520 to drive fluid substance(s) out of the surgical hand piece 520, through the mixer attachment 130, and out of a cannula 332 of the mixer attachment 130. Generally, the deformable basket 524 may facilitate more efficient and ergonomic handling of the hand piece 520, as the deformable basket 524 enables actuation of the hand piece 520 regardless of rotational orientation in the user's hand. Additional details with regard to the coupling of the deformable basket 524 and the drive system of the hand piece 520 are illustrated and described with reference to FIGS. 5B-5D.

As further depicted in FIG. 5A, the hand piece 520 includes a rotating endcap 522. The endcap 522 is configured to be removably screwed, or threaded, onto the proximal end 336 of the outer housing 310 to secure and/or exchange the gas canister 450 within the hand piece 520. Additionally, the endcap 522 functions as a mechanism for puncturing the seal of the nozzle 465 on the gas canister 450. For example, prior to or during use of the hand piece 520, the user can screw or rotate the endcap 522 to a most distal position, which in turn will act on the gas canister 450 and drive the gas canister 450 toward the puncture pin 458. Additional details with regard to the rotating endcap are illustrated and described with reference to FIGS. 5B-5D.

With reference now to FIGS. 5B-5D, the deformable basket 524 couples to the link 480 via a collar 528 slidably disposed within the primary chamber 424. In some embodiments, the collar 528 and the link 480 are monolithically formed as a single component. In some embodiments, the collar 528 and the link 480 are fixedly or removably coupled with one another. During use, a user compresses the lever arms 526 of the deformable basket 524, which causes the lever arms 526 to straighten and extend proximally into the primary chamber 424 against the collar 528. This, in turn, causes the collar 528 to slide proximally within the primary chamber 424 and act upon the link 480 and collar 478. Because the collar 478 engages with the flange 474 of the piston sleeve 466, the piston sleeve 466 is thereby also translated proximally, along with the connector 464, piston 468, and in some embodiments, the puncture pin attachment 470. Accordingly, the entire piston assembly 456 can be proximally translated upon compression of the deformable basket 524 to drive the injection of fluids from the hand piece 520 and into the mixer attachment 130.

Upon release of the deformable basket 524, the lever arms 526 automatically return to their resting, outwardly-curved state due to an elasticity of the lever arms 526. As the lever arms 526 return to their resting state, the lever arms 526 pull on the collar 528, which in turn pulls the piston assembly 456 to its resting, distal position.

As further shown in FIGS. 5B-5D, the gas canister 450 is at least partially supported within the drive chamber 428 via a gas canister sleeve 572, which is disposed circumferentially around at least a portion of the gas canister 450 to secure the gas canister 450 within the drive chamber 428. In some embodiments, the gas canister sleeve 572 is secured in place via one or more pins 574, which can be inserted through opening(s) 512 in the outer housing 310 and fastened into bore(s) 576 of the gas canister sleeve 572. In some embodiments, each pin 574 includes a screw that can be screwed or threaded into a corresponding thread in the bore(s) 576. In some embodiments, the one or more pins 574 are secured in place via a friction-fit with the bore(s) 576.

An inner surface at the proximal end of the gas canister sleeve 572 includes one or more threads 578 configured to mate with one or more corresponding outer threads 582 on an inner screw 580 disposed within, and fixedly coupled to, the endcap 522. To attach the endcap 522 onto the hand piece 520, the user may screw or thread the inner screw 580 of the endcap 522 into the proximal end of the gas canister sleeve 572 via the thread(s) 578 and 582. In some embodiments, as the inner screw 580 is threaded into the gas canister sleeve 572, the inner screw 580 is configured to contact the proximal end of the gas canister 450 such that the inner screw 580 drives the gas canister sleeve 572 distally and toward the puncture pin 458. At a certain point during threading of the inner screw 580, the seal on the nozzle 465 is pressed against the puncture pin 458, which punctures the seal and releases gas from the gas canister 450 (e.g., and into the conduit 475 of the puncture pin attachment 470) to pressurize the hand piece 520. Accordingly, the endcap 522 functions as a mechanism for puncturing the seal of the nozzle 465 on the gas canister 450, as noted above.

In some embodiments, a spring 560 or other biasing device is disposed circumferentially around the nozzle 465 of gas canister 450 and against the puncture pin attachment 470. The spring 560 biases the gas canister 450 proximally to prevent the gas canister 450 from freely moving in the distal direction through the drive chamber 428 prior to distal actuation by the endcap 522. Accordingly, the spring 560 can function to prevent the puncture pin 458 from prematurely puncturing the seal of the nozzle 465 during storage and/or handling of the hand piece 520. In some embodiments, the spring 560 includes a coil spring, though other types of spring are also contemplated.

FIG. 6A illustrates a cross-sectional view of an exemplary mixer attachment 600, according to embodiments described herein. Meanwhile, FIG. 6B illustrates an exploded view of the mixer attachment 600, according to embodiments described herein. For clarity, FIGS. 6A and 6B are herein described together.

Generally, the mixer attachment 600 is representative of the mixer attachment 130 described above. As depicted in FIG. 6A, the mixer attachment 600 includes a connector 602, a powder plate 604, a plurality of mixing plates 603 including one or more channeled plates 606 and one or more flat plates 608, at least one linear mixer 610, and a cannula 612. The mixer attachment 600 may be attached to a distal end of a hand piece (e.g., hand piece 100, hand piece 320, or hand piece 520) via mating of the connector 602 with a connector of the hand piece, such as a Luer lock, to create a leak-free fluidic seal therebetween.

The mixer attachment 600 further includes a front housing 614, a rear housing 616, and a sealing ring 618. The front housing 614 receives and permanently couples with the cannula 612 via a UV cured adhesive, molding, other binding agent, or other similar attachment mechanism, and the rear housing 616 connects to the hand piece (e.g., hand piece 100, hand piece 320, or hand piece 520) via the connector 602, which may be operable to couple to a threaded connector of the hand piece. In some embodiments, the front housing 614 and the rear housing 616 are configured to be removably coupled to each other through a threaded connection, such as a Luer-type connection. For example, the rear housing 616 may be configured to thread over the front housing 614. In other embodiments, the front housing 614 and the rear housing 616 may be snap-fit together. In some embodiments, the front housing 614 and the rear housing 616 are fixedly coupled to each other during assembly, such as by use of ultrasonic welding, laser welding, adhesive, bonding agent, or other similar techniques. When the rear housing 616 is attached to the front housing 614, the one or more flat plates 608 and one or more channeled plates 606 (e.g., altogether referred to as mixing plates 603) are configured to be disposed in a central opening 615 of the front housing 614 and pressed against each other.

The front housing 614 and rear housing 616 may be manufactured such that a plurality of flat plates 608 and channeled plates 606 may be contained within the mixer attachment 600 when the front housing 614 and the rear housing 616 are coupled together. In some embodiments, for example, the front housing 614 and the rear housing 616 may be configured to contain 4, 5, or 6 mixing plates 603 (e.g., two or three of each of flat plates 608 and/or channeled plates 606) therebetween. In the embodiment shown, two flat plates 608 and three channeled plates 606 are shown.

In some embodiments, when the housings 614 and 616 are coupled together, i.e., when the mixer attachment 600 is in an assembled state, the flat plates 608 and channeled plates 606 may be secured in position by inward pressure or “squeezing” forces created by the connection of the front housing 614 and the rear housing 616 to each other; in such embodiments, the flat plates 608 and channeled plates 606 may not be fixedly attached to the front housing 614 or rear housing 616. In certain other embodiments, however, the flat plates 608 and/or channeled plates 606 may be fixedly attached to the front housing 614 (and/or rear housing 616) and not reliant on the inward squeezing forces created by the coupling of the front housing 614 and rear housing 616 to secure the flat plates 608 and channeled plates 606 to one another.

In some embodiments, the mixer attachment 600 includes the sealing ring 618 disposed between the front housing 614 and the rear housing 616 when the mixer attachment 600 is assembled. The sealing ring 618 may create a leak-free seal between an outer surface of the front housing 614 and an inner surface of the rear housing 616 to provide retaining of gas inside the mixer attachment 600 during use. In some embodiments, as shown in FIG. 6B, the sealing ring 618 may be configured to fit within a pocket or space of the rear housing 616, or may be configured to fit within a pocket or space of the front housing 614. In some embodiments, the sealing ring 618 may include an elastomeric O-ring or similar device.

In some embodiments, the mixer attachment 600 includes a powder plate 604 configured to be disposed at a proximal end (e.g., closest to the connector 602) of the mixer attachment 600. The powder plate 604 may have a cup-like or a disc-like shape and may be configured to support, or contain, one or more mesh and/or membrane filter layers and a powder substance 622 for mixing (e.g., the second substance described herein). In the example of FIGS. 6A and 6B, an inlet mesh layer 623 and an outlet mesh layer 621 are shown. The inlet mesh layer 623 is configured to be arranged proximally to the powder substance 622 during use and prevents the powder substance 622 from travelling proximally through the mixer attachment 600. Meanwhile, the outlet mesh layer 621 is configured to be arranged distal to the powder substance 622 during use and prevents the powder substance 622 from travelling distally through the mixer attachment 600 prior to a fluid substance being flowed through mixer attachment 600 during injection/application of a retinal patch. The inlet mesh layer 623 and the outlet mesh layer 621 may, therefore, have pores arranged and/or sized to retain the powder substance 622 within the powder plate 604 until the fluid substance is passed through the powder plate 604, which mixes and at least partially dissolves the powder substance 622. Once the powder substance 622 is at least partially dissolved in the fluid substance, the “combined substance” can travel or flow through the outlet mesh layer 621 and powder plate 604 and through the remainder of the mixer attachment 600. In some embodiments, the inlet mesh layer 623 and/or outlet mesh layer 621 have pores between 40-100 micrometers in size. In some embodiments, the inlet mesh layer 623 and the outlet mesh layer 621 have pores of the same size; in some embodiments, the inlet mesh layer 623 and outlet mesh layer 621 have pores of different sizes. In some embodiments, more than two mesh and/or membrane filter layers may be arranged in the powder plate 604 during use.

In some embodiments, the rear housing 616 and/or connector 602 includes a diffuser to diffuse, or spread, the flow of the first fluid substance towards a perimeter of the powder plate 604 during use.

Distal to the powder plate 604, the mixer attachment 600 may include any suitable number and/or arrangement of mixing plates 603 configured to cause a combined substance (e.g., a combination of the first fluid substance and a second substance, such as the powder substance 622) being translated through the mixer attachment 600 to be homogenously mixed. For example, in some embodiments, the mixer attachment 600 may include three channeled plates 606 alternated with two flat plates 608, and a linear mixer 610. In certain other embodiments, the mixer attachment 600 may include only one channeled plate 606 disposed between two flat plates 608, and a linear mixer 610; or, one flat plate 608 disposed between two channeled plates 606, and a linear mixer 610. Generally, the linear mixer 610 may be configured to be coupled with, or disposed adjacent to, the distal-most mixing plate 603 in the mixer attachment 600. In the example shown, the linear mixer is shown as being rigidly or removably attached to the most distal channeled plate 606. However, other arrangements are also contemplated.

To thoroughly mix the combined substance and create a homogenous mixture or near-homogenous mixture, the flat plates 608 and channeled plates 606 may be alternated to create a turbulent path for the combined substance as it is forced, or passed, through the mixer attachment 600. In one particular arrangement, as shown in FIG. 6B, after the first fluid substance is distally passed through the powder plate 604 (and inlet mesh layer 623) to combine with the second powder substance 622, the combined substance first comes into contact with a proximal side of a channeled plate 606 (after passing through outlet mesh layer 621). The proximal side of the channeled plate 606 includes a plurality of surface channels formed therein that guide the combined substance radially outward toward a plurality of circumferential openings in the channeled plate 606. The combined substance is passed through the openings, after which the combined substance contacts a flat plate 608 disposed distal to the channeled plate 606. A flat proximal side surface of the flat plate 608, in combination with another plurality of surface channels formed in a distal side of the first channeled plate 606, forces the combined substance to flow radially inward and toward a central opening in the flat plate 608.

Thereafter, the combined substance is passed through the central opening of the flat plate 608, before coming into contact with the proximal side of another channeled plate 606, and so on, until the combined substance is passed through the linear mixer 610 and into the cannula 612. The linear mixer 610 may include a rod with a plurality of channels 620 etched in alternating circumferential arcs along a length of the rod. The plurality of surface channels 620 in the linear mixer 610 may be configured to create converging and diverging streams, or flow paths, of the combined substance for efficient mixing thereof as it is directed to the cannula 612 at the distal end of the mixer attachment 600.

The alternating pattern of channeled plates 606 and flat plates 608, in combination with the surface channels of the channeled plates 606 and linear mixer 610, creates a turbulent flow path for the combined substance as it passes through the mixer attachment 600, thereby facilitating homogenous or near-homogenous mixing of the combined substance.

Generally, to force the combined substance to pass through the openings and surface channels of the channeled plates 606 and flat plates 608, the plates 606 and 608 may be tightly toleranced with the central opening 615 of the front housing 614 (and/or rear housing 616, in some embodiments), such that an outer diameter of the plates 606 and 608 is substantially the same as an inner diameter of the central opening 615. Accordingly, any fluids passing through the mixer attachment 600, such as the combined substance, are forced to pass through the openings and surface channels of the channeled plates 606 and flat plates 608 and cannot creep around the outer edges of the plates 606 and 608.

As further shown in FIG. 6A, the mixer attachment 600 further includes a needle guard 624 in some embodiments. The needle guard 624 is configured to attach to the front housing 614 and sheath the cannula 612 during assembly and storage. Generally, the needle guard 624 may attach to the front housing 614 via any suitable attachment mechanism, such as by a threaded connection, a snap-fit connection, or by friction fit. In the example shown, the needle guard 624 includes one or more bearings 626 configured to mate with grooves or other features on the front housing 614 to create a snap-fit between the two components.

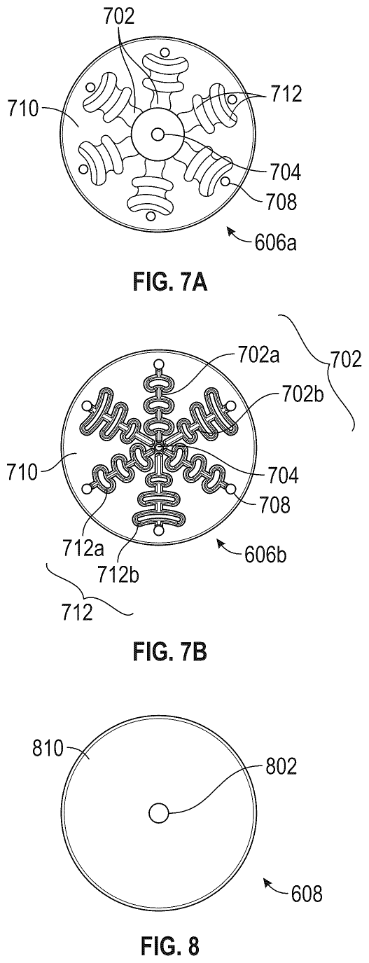

FIG. 7A illustrates a proximal side view of an exemplary channeled plate 606a of the mixer attachment 600, according to embodiments described herein. The channeled plate 606a is an exemplary embodiment of the channeled plates 606 described above. As described with reference to FIGS. 6A-6B, the channeled plate 606a may include surface channels (e.g., 702 and 712, described in further detail below) formed on one or both sides of the channeled plate 706a. Though only a proximal side 710 of the channeled plate 606a is illustrated in FIG. 7A, a distal side of the channeled plate 606a may be a mirror image of the proximal side 710 or have a different pattern of surface channels formed therein. In some embodiments, the proximal side 710 or the distal side of channeled plate 606a may have a generally flat surface.

As discussed in reference to FIGS. 6A-6B, since the channeled plates 606 and flat plates 608 are pressed against each other within mixer attachment 600 by squeezing forces between the front housing 614 and the rear housing 616, and since the channeled plates 606 and flat plates 608 are tightly toleranced with the central opening 615, a combined substance travels through channels created in the proximal side and/or distal side of the channeled plates 606 to reach through holes in the channeled plate 606 for passing through the mixer attachment 600.

In the depicted embodiment, the channeled plate 606a comprises a solid center 704, where the substances to be mixed (e.g., the first fluid substance and the second substance) may first come into contact with the proximal side of the channeled plate 606a as the substance is passed through mixer attachment 600 during mixing. After the substance contacts the center 704, the substances are forced (e.g., guided) radially outward through a plurality of radially-extending surface channels 702 (six are shown in FIG. 7A), and one or more laterally-extending and rib-shaped surface channels 712 intersecting with the one or more of the radially-extending surface channels 702 (two channels 712 are shown intersecting with each channel 702 in FIG. 7A), towards the outer circumference of the channeled plate 606a. The surface channels 702 and 712 create converging and diverging flow paths for the flowing substances, thereby causing the first fluid substance to be mixed with the second substance.

When the substance reaches the through holes 708 along the outer circumference of the channeled plate 606a, the substance passes through the through holes 708 and comes out on the distal side of the channeled plate 606a. Once on the backside, the substance is obstructed by a downstream flat plate 608, which in combination with surface channels on the distal side of the channeled plate 606a, causes the substance to flow radially inward towards the center of the channeled plate 606a before passing through a center opening 802 of the downstream flat plate 608. Note that any number and arrangement of surface channels 702 and/or 712 may be formed in the proximal side and/or distal side of a channeled plate 606a. Further, in certain other embodiments, a distal side of a channeled plate 606a may have a different pattern of surface channels 702 and/or 712 as compared to the proximal side thereof.

In the embodiment shown, each radially-extending surface channel 702 intersects with the same number and arrangement of laterally-extending surface channels 712. More particularly, each surface channel 702 intersects with two surface channels 712, wherein the most radially outward of the two surface channels 712 is longer in length than the most radially inward of the two surface channels 712. Additionally, each of the surface channels 712 is bowed, or curved, in the same direction relative to the center 704. Accordingly, the arrangement of surface channels 702 and surface channels 712 in FIG. 7A, may be described as being “uniform.”

FIG. 7B illustrates a proximal side view of another exemplary channeled plate 606b of the mixer attachment 600, according to embodiments described herein. The channeled plate 606b is another exemplary embodiment of the channeled plates 606 described above. Similar to the example in FIG. 7A, the channeled plate 606b may include surface channels formed on one or both sides of the channeled plate 606b. Though only the proximal side 710 of the channeled plate 606b is illustrated in FIG. 7B, a distal side of the channeled plate 606b may be a mirror image of the proximal side 710 or have a different pattern of surface channels formed therein. In some embodiments, the proximal side 710 or the distal side of channeled plate 606b may have a generally flat surface.

The channeled plate 606b is substantially similar to the channeled plate 606a but for the arrangement of the surface channels 702 and the surface channels 712, which form a “non-uniform” pattern. In the example shown, each radially-extending surface channel 702 intersects with the same number of laterally-extending surface channels 712. However, the size and/or arrangement of the surface channels 712 varies between adjacent surface channels 702. For example, of the six radially-extending surface channels 702 that are depicted, three surface channels 702a are each intersected by three surface channels 712a having uniform flow path lengths and curvatures. The surface channels 712a are generally short and curved outward relative to the center 704. The remaining three surface channels 702b, which are alternated with the surface channels 702a around the center 704, are intersected by three surface channels 712b having similar curvatures but different flow path lengths. For example, surface channels 712b all curve inward relative to the center 704, but increase in length with increased distance from the center 704. This general arrangement of channels with different lengths facilitates the staggered, or variable, mixing of the first fluid substance and the second substance at different times during the passing of the first fluid substance through the mixer attachment 600. In other words, the mixing of the two substances may be spread out, or drawn out, as a result of the different lengths of the channels, which may facilitate a more homogenous mixture by causing a “folding” type of mixing behavior where unmixed substance(s) can meet with previously mixed substance(s).

In some embodiments, the channeled plate 606a or 606b may be formed of plastic or metallic materials. In some embodiments, the channeled plate 606a or 606b may be formed of an elastomeric material. In some embodiments, the channeled plate 606a or 606b may be 3D-printed, molded, machined, or etched (e.g., chemically or physically etched, for example) to include various patterns and/or directional paths from the center. However, other materials, methods of manufacture, and/or arrangements are also contemplated for the channeled plates 606a and 606b.

FIG. 8 illustrates an exemplary flat plate 608 of the mixer attachment 600, according to embodiments described herein. As described in reference to FIGS. 6A-6B, the flat plate 608 may have generally flat proximal and/or distal side surfaces and be positioned in the mixer attachment 600 in an alternating configuration with channeled plates 606. Though only a proximal side 810 of the flat plate 608 is illustrated in FIG. 8, a distal side of the flat plate 608 may be a mirror image of the proximal side 810. In some embodiments, however, the proximal side 810 or distal side may have a pattern of surface channels formed therein, similar to channeled plates 606.

As shown in FIG. 8, the flat plate 608 further includes a center opening 802, through which the combined substance or other fluids may pass through the flat plate 608 during mixing thereof. Again, since the channeled plates 606 and flat plates 608 are pressed against each other within mixer attachment 600 by squeezing forces between the front housing 614 and the rear housing 616, and since the channeled plates 606 and flat plates 608 are tightly toleranced with the central opening 615, a combined substance travels through the center opening 802 of the flat plate 608 to reach the distal side of the flat plate 608 when passing through the mixer attachment 600. Thus, the flat surface of the proximal side 810 creates a blockade, or flow restriction, that forces fluids through the center opening 802 during mixing, thereby directing the flow of fluids to the center of downstream channeled plates 606.

In some embodiments, the flat plate 608 may be formed of a plastic or metallic material, for example. In some embodiments, the flat plate 608 may be formed of an elastomeric material. The flat plate 608 may be 3D-printed, machined, or molded to include the center opening 802, but otherwise have a smooth surface.

FIG. 9A illustrates a perspective view of the powder plate 604, according to embodiments described herein. FIG. 9B illustrates a side cross-sectional view of the powder plate 604, according to embodiments described herein. Meanwhile, FIG. 9C illustrates a perspective view of the powder plate 604, in addition to the outlet mesh layer 621, the inlet mesh layer 623, and the powder substance 622 configured to be disposed within the powder plate 604 during use, according to embodiments described herein. For clarity, FIGS. 9A-9C are herein described together.

As shown, the powder plate 604 has a cup-like shape and includes a base 902 attached to an annular and/or tubular wall 904. The base 902 and wall 904 form a cavity 906 within which the one or more mesh and/or membrane filter layers and second substance can be disposed in for use. For example, as shown in FIG. 9C, the outlet mesh layer 621, the inlet mesh layer 623, and the powder substance 622 can be arranged within the cavity 906 during use. In such embodiments, the outlet mesh layer 621 can be positioned nearest to the base 902, the inlet mesh layer 623 can be arranged furthest from the base 902 (and proximal to the outlet mesh layer 621), and the powder substance 622 can be disposed and confined between the inlet mesh layer 623 and the outlet mesh layer 621 until it is combined with the first fluid substance. Upon combination of the powder substance 622 with the first fluid substance, the resulting combined substance can pass through the outlet mesh layer 621 and through an opening 908 formed in the base 902 before reaching the mixing plates 603 for thorough mixing. Note that although shown as a disc or puck in FIG. 9C, the powder substance 622 may generally include loose, granular powder rather than lumped or caked material.

In some embodiments, a proximal surface 910 of the base 902 includes a plurality of features 912 formed thereon for supporting the one or more mesh and/or membrane filter layers disposed within the powder plate 604. For example, the plurality of features 912 may contact and support the outlet mesh layer 621, which may press against the features 912 during use. In such embodiments, the plurality of features 912 prevent the outlet mesh layer 621 (and/or other mesh and/or membrane filter layers) from leaning or pressing against the proximal surface 910, which could otherwise obstruct the combined substance from flowing through the outlet mesh layer 621 and toward/into the opening 908. In other words, the features 912 facilitate the unobstructed flow of the combined substance to the opening 908. In some embodiments, the features 912 include protrusions, such as mesa-like protrusions, that extend proximally from the proximal surface 910. In certain other embodiments, however, the proximal surface 910 can include grooves or channels formed therein; in such embodiments, the proximal surface 910 can support the outlet mesh layer 621 (and/or other mesh and/or membrane filter layers) while the combined surface passes through the outlet mesh layer 621 and flows to the opening 908 via the grooves or channels.

As shown in FIGS. 9A and 9C, in some embodiments, an outer surface 914 of the wall 904 includes one or more locking and/or alignment features 916 for securing and/or aligning the powder plate 604 in the central opening 615 of the front housing 614.

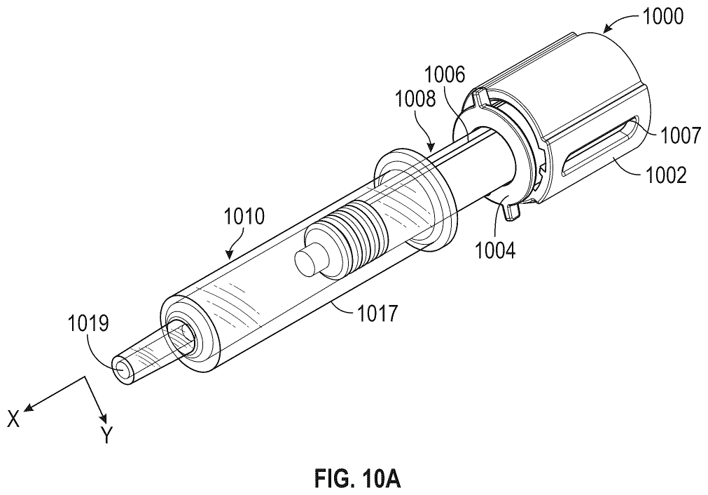

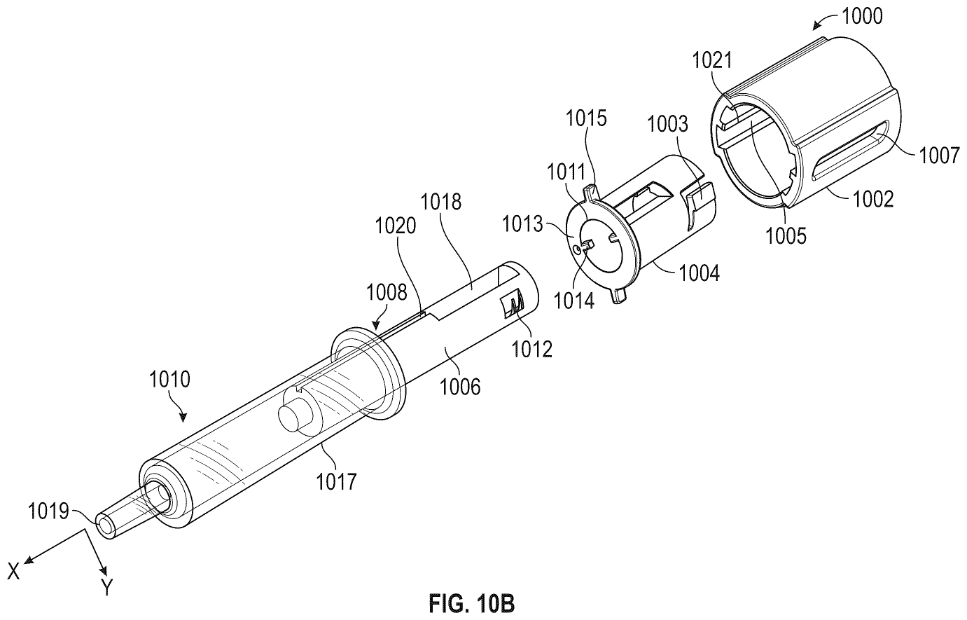

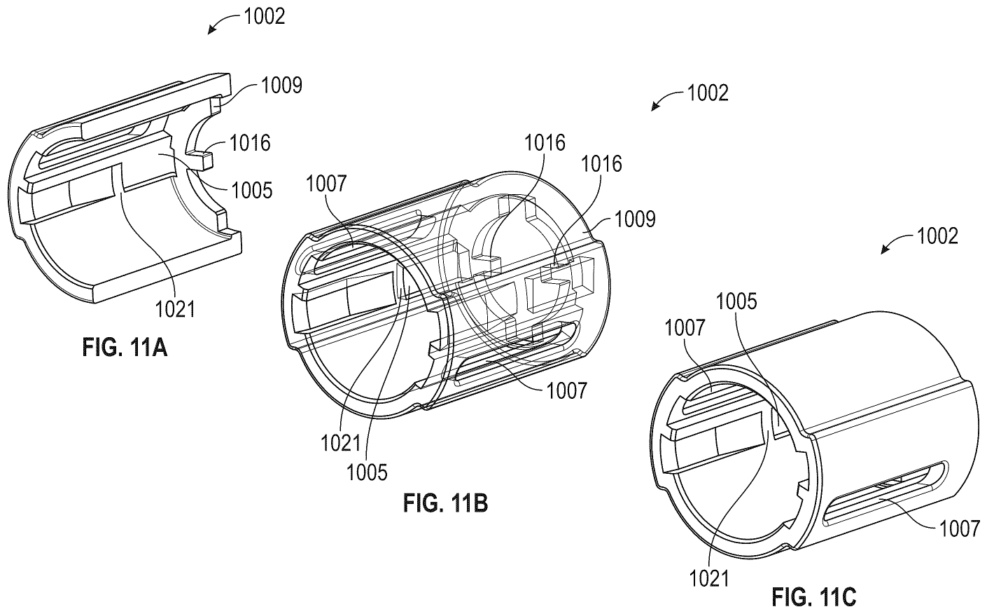

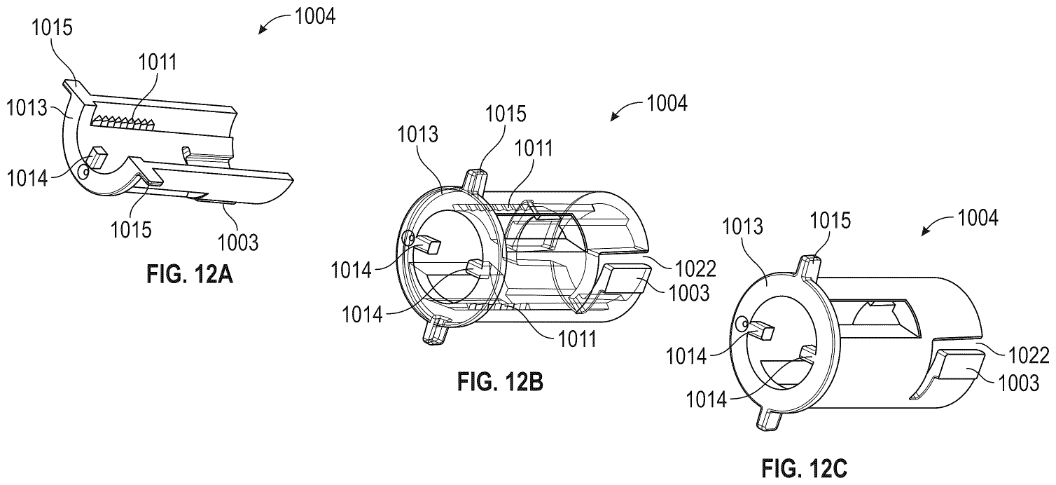

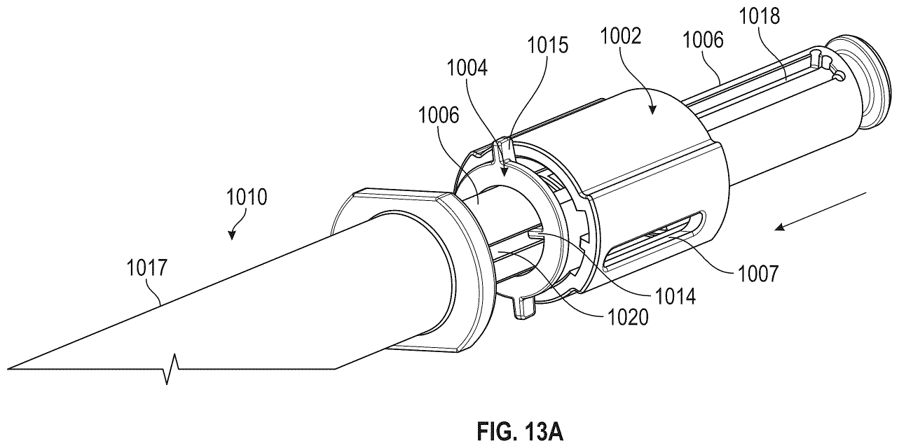

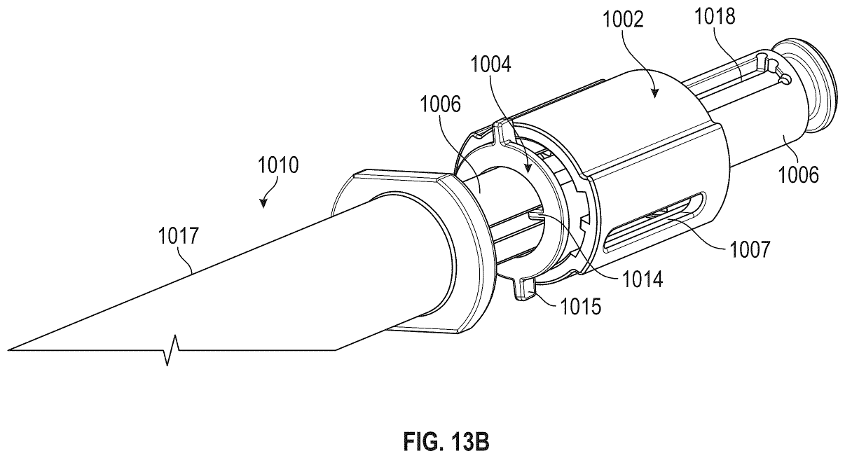

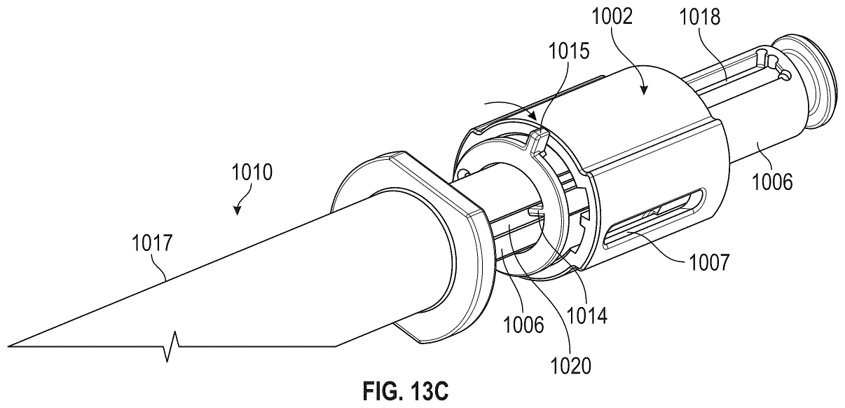

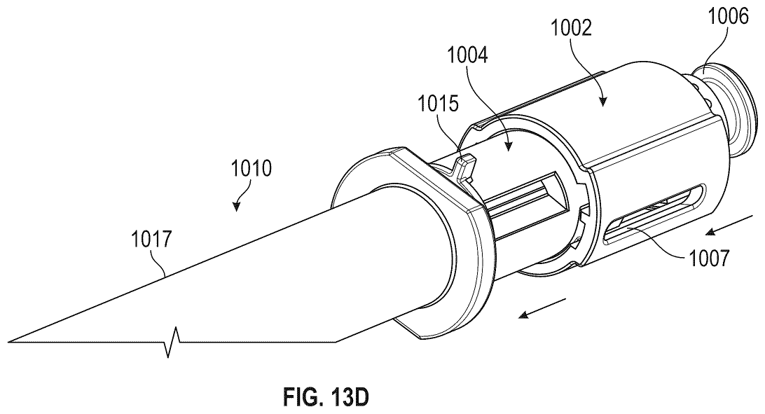

FIG. 10A illustrates a perspective view of an exemplary variable dose stop mechanism 1000, according to embodiments herein. FIG. 10B illustrates an exploded view of the variable dose stop mechanism 1000, according to embodiments herein. FIGS. 11A-11C illustrate a perspective cross-sectional view, a perspective phantom view, and a perspective view of a lock ring of the variable dose stop mechanism 1000, while FIGS. 12A-12C illustrate a perspective cross-sectional view, a perspective phantom view, and a perspective view of a dose ring of the variable dose stop mechanism 1000, respectively, according to embodiments herein. FIGS. 13A-13D illustrate various positions of the exemplary variable dose stop mechanism 1000 during administration of two successive doses, according to embodiments herein. For clarity, FIGS. 10A-10B, 11A-11C, 12A-12C, and 13A-13D are described together herein.

Generally, the dose stop mechanism 1000 is an example embodiment of dose stop mechanisms 218 and 448 illustrated in FIGS. 2, 4A-4D, and 5B-5D. The dose stop mechanism 1000 ensures an appropriate volume of a first fluid substance is delivered through a mixer attachment (e.g., mixer attachment 600) for mixing with an amount (e.g., a predetermined amount) and type of solid substance in the mixer attachment, without the need for manual measurement of the first fluid substance. Delivering an appropriate volume of a first fluid to be mixed with a given amount of the solid substance ensures the combined substance has one or more desired properties to allow for timely curing of the combined substance and appropriate treatment effect for the patient. In some embodiments, the dose stop mechanism 1000 further provides the provision of multiple, substantially equal volumes of the first fluid substance to a mixer attachment for dispensing of multiple doses with a single syringe or cartridge.

The dose stop mechanism 1000 generally includes a lock ring 1002 and a dose ring 1004, which are both generally tubular in shape. The lock ring 1002 fixedly couples to an interior of the outer housing of a hand piece (e.g., outer housing 310 of hand piece 320) and movably secures the dose ring 1004 within the hand piece. The dose ring 1004, meanwhile, nests within the lock ring 1002 as shown in FIG. 10A and is configured to rotate and translate axially within the lock ring 1002 in a limited or controlled manner. The dose ring 1004 further engages with a proximal end of a plunger rod 1006 of a plunger 1008 disposed within a surgical hand piece (e.g., hand pieces 100, 320, or 520) to guide movement of the plunger 1008 for administration of separate doses. As described with reference to FIGS. 2 and 4A-4D, a distal end of the plunger 1008 is arranged within and operable to longitudinally (i.e., linearly) translate through a barrel 1017 of a syringe 1010 towards a distal end of the hand piece. Distal translation of the plunger 1008 causes a first fluid substance contained within the barrel 1017 to be expelled from a discharge orifice 1019 at the distal end of the syringe 1010.

As shown in FIGS. 10B and 11A-11C, an inner surface of the lock ring 1002 includes a detention feature 1005 with a raised lip 1021 that is configured to engage with a key 1003 of the dose ring 1004 to secure the dose ring 1004 in place during the administration of a first dose. Generally, the detention feature 1005 is sized and shaped to receive the key 1003, while the raised lip 1021 prevents distal translation of the key 1003 and thus, the dose ring 1004, through the lock ring 1002 prior to and during administration of the first dose. Adjacent to the detention feature 1005 is a keyway 1007 that extends along at least the inner surface of the lock ring 1002. Generally, the keyway 1007 includes a channel that runs parallel to a longitudinal axis X of the hand piece and/or dose stop mechanism 1000. After administration of the first dose, the dose ring 1004 can be rotated such that the key 1003 is aligned with, and/or received in the keyway 1007, which enables the dose ring 1004 to be translated distally through the lock ring 1002 during administration of a second dose.

Also shown in FIG. 11B, the lock ring 1002 may include a collar 1009 disposed at a proximal end thereof and having one or more alignment features 1016 extending radially inward therefrom. The alignment features 1016 may engage with one or more corresponding alignment features 1018 on the plunger rod 1006 to facilitate rotational alignment of the plunger 1008 with the lock ring 1002 while it longitudinally translates during administration of the first and/or second dose.

As shown in FIGS. 10B and 12A-12C, the dose ring 1004 includes the key 1003, which is configured to engage with the detention feature 1005 on the lock ring 1002 during administration of the first dose and with the keyway 1007 during administration of the second dose. Generally, the key 1003 includes a protrusion extending from an outer surface of the dose ring 1004 that is suitably shaped and/or sized for receipt in the detention feature 1005 and keyway 1007. In some embodiments, the key 1003 is at least partially defined by a cutout 1022, which allows the key 1003 to deflect inwards to facilitate transitioning between the detention feature 1005 and the keyway 1007. In such embodiments, the detention feature 1005 may include a sloped surface that requires the key 1003 to deflect inwards in order to transition between the detention feature 1005 and the keyway 1007 during rotation of the dose ring 1004 within the lock ring 1002. The sloped surface may function to prevent unintentional rotation of the dose ring 1004 during use and involves a rotational force provided by the user to cause the key 1003 to deflect inwards and allow transition of the key 1003 between the retention feature 1005 and the keyway 1007.

In addition to the key 1003, the dose ring 1004 further includes teeth 1011 extending from an inner surface of the dose ring 1004 near the distal end of the dose ring 1004. The teeth 1011 are configured to engage with corresponding teeth 1012 disposed on the proximal end of the plunger rod 1006 during administration of the second dose. Also at the distal end of the dose ring 1004 is a collar 1013 having alignment features 1014 and 1015 extending radially therefrom. The collar 1013 functions as a stop for the plunger 1008 at the conclusion of the first dose, while the alignment features 1014, which extend radially inward from the collar 1013, are configured to permanently engage with alignment features 1020 on the plunger rod 1006 to maintain rotational alignment between the plunger 1008 and dose ring 1004 during administration of both the first dose and the second dose. The alignment features 1015, which extend radially outward from the collar 1013, are configured to interact with one or more corresponding alignment features, such as channels, formed in a syringe sleeve surrounding the syringe barrel 1017, such as the syringe sleeve 420, to maintain rotational alignment of the dose ring 1004 while it translates longitudinally during administration of the second dose.

Turning now to FIGS. 13A-13D, the actuation of the dose stop mechanism 1000 during the administration of two successive doses of the first fluid substance is shown. In FIGS. 13A and 13B, the dose ring 1004 is disposed in a first rotational orientation corresponding to the first dose. In this first rotational orientation, the teeth 1011 on the dose ring 1004 are not aligned or engaged with the corresponding teeth 1012 of the plunger rod 1006. In this orientation, the plunger 1008 is free to translate distally through the dose ring 1004 independent of movement of the dose ring 1004, as guided by the alignment features 1014 of the dose ring 1004 that are permanently engaged with the alignment features 1020 of the plunger rod 1006.

In the first rotational orientation, the key 1003 of the dose ring 1004 is also disposed in the detention feature 1005 of the lock ring 1002, and the raised lip 1021 of the detention feature 1005 prevents the dose ring 1004 from translating longitudinally and axially through the lock ring 1002. As a result, the dose ring 1004 is “locked” in its longitudinal position while disposed in the first rotational orientation. Accordingly, upon actuation of the drive system in the hand piece (e.g., upon pressurization of the hand piece via release of gases from the gas canister 450), the plunger 1008 can translate distally through the dose ring 1004 to dispense the first dose of the first fluid substance from the syringe 1010, while the dose ring 1004 remains locked in position within the lock ring 1002. Distal translation of the plunger 1008 is shown between FIGS. 13A and 13B.

The plunger 1008 translates distally through the dose ring 1004 while the dose ring 1004 is in the first rotational orientation until the teeth 1012 on the plunger rod 1006 come into contact with the collar 1013. The collar 1013 thus functions as a stop for the plunger 1008 at the conclusion of the first dose. At this point, the first dose has been dispensed (e.g., into the mixer attachment 600), and the plunger 1008 is “locked” in place until a second dose is actuated.

In some embodiments, in the first rotational orientation, the dose ring 1004 is operable to allow the plunger 1008 to move freely and distally through the dose ring 1004 up until a first predetermined axial distance, at which point the teeth 1012 come into contact with the collar 1013 on the dose ring 1004 to prevent further movement of the plunger 1008 and further dispensing. The first predetermined distance may correspond with a first predetermined volume (e.g., 0.50-1.0 milliliters (mL), such as 0.75 mL) of the first fluid substance in the syringe 1010, which is flowed through the mixer attachment 600 to mix with the second substance contained in the powder plate 604 upon the distal movement of the plunger 1008. In some embodiments, the first predetermined volume is measured to facilitate a desired ratio of first substance to second substance in the first dose when the first and second substances are mixed, and/or a desired density of the mixture.

In FIG. 13C, the dose ring 1004 is rotated to a second rotational orientation. In this second rotational orientation, the key 1003 is moved out of the detention feature 1005 and received in the keyway 1007 that runs parallel to the major longitudinal axis X. As a result, the key 1003 is no longer detained by the raised lip 1021, and the dose ring 1004 is able to translate longitudinally through the lock ring 1002.

In addition, rotation of the dose ring 1004 to the second rotational orientation causes the teeth 1011 of the dose ring 1004 to align and “catch,” or interlock with, the teeth 1012 of the plunger rod 1006. In this state, the plunger 1008 is secured to the dose ring 1004 via the engagement of the teeth 1011 and 1012, and longitudinal movement of the dose ring 1004 will cause similar movement of the plunger 1008.

As shown in FIG. 13D, after rotation of the dose ring 1004 to the second rotational orientation, which frees the dose ring 1004 from the detention feature 1005 and secures the plunger 1008 to the dose ring 1004, the user may again actuate the drive system of the hand piece (e.g., via actuation of the lever 324 or deformable basket 524) to drive the dose ring 1004 and plunger 1008 further distally. This coordinated movement of the dose ring 1004 and the plunger 1008 causes the syringe 1010 to dispense the second dose of the first fluid substance therefrom. The dose ring 1004 and plunger 1008 can move in tandem, uninhibited by the lock ring 1002, for a specified longitudinal distance to deliver the second dose of a desired volume of the first fluid substance from the syringe 1010. Generally, the dose ring 1004 and the plunger 1008 may be driven distally by the drive system until the alignment features 1015 on the dose ring 1004 make contact with stops formed in a syringe sleeve surrounding the syringe barrel 1017, such as the syringe sleeve 420, thereby concluding the administration of the second dose.

In some embodiments, in the second rotational orientation, the dose ring 1004 and the plunger 1008 can move freely and distally in tandem through the hand piece up until a second predetermined axial distance. At this point, the dose ring 1004 and/or plunger 1008 can come into contact with a stop disposed adjacent to a proximal end of the syringe 1010 in the hand piece to prevent further movement of the dose ring 1004 and plunger 1008 and further dispensing. The second predetermined distance may correspond with a second predetermined volume (e.g., 0.50-1.0 milliliters (mL), such as 0.75 mL) of the first fluid substance in the syringe 1010, which is flowed through the mixer attachment 600 to mix with the second substance contained in the powder plate 604 upon the distal movement of the plunger 1008. In some embodiments, the second predetermined volume is substantially equal to the first predetermined volume; in certain other embodiments, the first and second predetermined volumes are different. Generally, the second predetermined volume is measured to facilitate a desired ratio of first substance to second substance in the second dose when the first and second substances are mixed, and/or a desired density of the mixture.

As noted above, in some embodiments, the dose ring 1004 is engaged with a syringe sleeve (e.g., syringe sleeve 420) disposed around the syringe 1010. In such embodiments, rotation of the dose ring 1004 between the first rotational orientation and the second rotational orientation can be caused and/or facilitated by rotation of the syringe sleeve. For example, after a first dose or volume is dispensed from the syringe 1010 and through a first mixer attachment 600, the user may remove the first mixer attachment 600 from the hand piece and attach a second mixer attachment 600 thereto. As noted above, in some embodiments, the syringe sleeve can provide a threaded connector for coupling the first and second mixer attachments 600 to the syringe 1010. Accordingly, when attaching the second mixer attachment 600 onto the syringe 1010 via the threaded connector, the user will rotate the second mixer attachment 600, causing the syringe sleeve, as well as the dose ring 1004 coupled thereto, to rotate from the first rotational orientation to the second rotational orientation. In some embodiments, however, the dose ring 1004 may be rotated mechanically, automatically, and/or electromechanically by the user, an electromechanical motor, or another actuator disposed within a hand piece.

In some embodiments, the dose stop mechanism 1000 may comprise locking mechanisms to ensure a user (e.g., surgeon) is unable to mate the teeth 1011 of the dose ring 1004 with the teeth 1012 of the plunger rod 1006 prior to the connection of a second mixer attachment 600. Such locking mechanisms may comprise one or more mechanical features on the plunger rod 1006, such as protrusions, teeth, etc.

Generally, the various components of the devices of the present disclosure can be individually fabricated by any suitable molding and/or machining techniques (such as molding followed by secondary machining of plastic material) and/or three-dimensional (3D) printing techniques (such as 3-D printing of plastic material), and thereafter adhered or fit or otherwise coupled together during assembly. Alternatively, devices of the present disclosure can be monolithically molded and/or 3D printed.

The many features and advantages of disclosure are apparent from detailed specification, and, thus, it is intended by appended claims to cover all such features and advantages of disclosure which fall within scope of disclosure. Further, since numerous modifications and variations will readily occur to those skilled in art, it is not desired to limit disclosure to exact construction and operation illustrated and described, and, accordingly, all suitable modifications and equivalents may be resorted to that fall within scope of disclosure.

What is claimed is:

1. A mixer attachment for use with a surgical hand piece, the mixer attachment comprising:
a powder plate, the powder plate configured to comprise a solid substance for combining with a liquid substance to form a combined substance; and
a plurality of mixing plates configured to mix the solid substance and liquid substance in the combined substance, the plurality of mixing plates comprising:
one or more channeled plates, the one or more channeled plates each comprising one or more channels and one or more through holes configured to facilitate diverging and converging flow of the combined substance during mixing; and
one or more flat plates, the one or more flat plates each comprising an opening to direct flow of the combined substance between the one or more channeled plates.

2. The mixer attachment of claim 1, wherein at least one of the one or more channeled plates comprises a non-uniform pattern of the one or more channels.

3. The mixer attachment of claim 1, further comprising a mixer disposed adjacent to the plurality of mixing plates and configured to direct the combined substance to a distal end of the mixer attachment, wherein the mixer comprises a rod comprising one or more channels configured to facilitate mixing of the combined substance as it passes to the distal end of the mixer attachment.

4. The mixer attachment of claim 3, wherein the one or more channels of the mixer are configured to facilitate diverging and converging flow of the combined substance during mixing.

5. The mixer attachment of claim 1, wherein the powder plate is configured to contain at least one filter for preventing the solid substance from traveling through the mixer attachment prior to mixing with the liquid substance.

6. The mixer attachment of claim 1, wherein the mixer attachment is configured to couple to a distal end of an injector hand piece.

7. The mixer attachment of claim 6, wherein:
the injector hand piece comprises a plunger and a syringe,
the syringe is configured to contain the liquid substance prior to mixing, and
actuation of the plunger causes the liquid substance to be flowed into the mixer attachment for mixing when the mixer attachment is attached to the injector hand piece.

8. The mixer attachment of claim 7, wherein:
the injector hand piece is configured to be fluidically coupled to a pneumatic system of a surgical console; and
actuation of the plunger is pneumatically driven via the pneumatic system.

9. The mixer attachment of claim 7, wherein:
the injector hand piece comprises an internal drive mechanism; and
actuation of the plunger is driven via the internal drive mechanism.

10. The mixer attachment of claim 9, wherein the internal drive mechanism comprises a pre-charged pneumatic drive system.

11. A surgical hand piece, comprising:
a syringe, the syringe comprising a connector at a distal end of the syringe;
a plunger, the plunger coupled to a drive system and configured to translate toward the distal end of the syringe upon actuation of the plunger; and
a mixer attachment removably coupled to the connector at the distal end of the syringe.

12. The surgical hand piece of claim 11, wherein:
the surgical hand piece comprises a tethered hand piece,
the tethered hand piece comprises a connection port at a proximal end of the tethered hand piece for fluidly coupling the tethered hand piece with a surgical console, and
the surgical console comprises a pneumatic drive system for driving the plunger towards the distal end of the syringe.

13. The surgical hand piece of claim 11, wherein:
the surgical hand piece comprises an untethered hand piece, and
the untethered hand piece comprises an internal drive mechanism for driving the plunger towards the distal end of the syringe.

14. The surgical hand piece of claim 13, wherein the internal drive mechanism comprises a pre-charged pneumatic drive system.

15. The surgical hand piece of claim 11, wherein the mixer attachment comprises:
a powder plate configured to contain a solid substance for combining with a liquid substance to form a combined substance; and
a plurality of mixing plates configured to mix the solid substance and the liquid substance in the combined substance.

16. The surgical hand piece of claim 15, wherein the plurality of mixing plates comprises:
one or more channeled plates, the one or more channeled plates each comprising one or more channels and one or more through holes configured to facilitate diverging and converging flow of the combined substance during mixing; and
one or more flat plates, the one or more flat plates each comprising an opening to direct flow of the combined substance between the one or more channeled plates.

17. The surgical hand piece of claim 16, wherein at least one of the one or more channeled plates comprises a non-uniform pattern of the one or more channels.

18. The surgical hand piece of claim 16, wherein the mixer attachment further comprises a mixer disposed adjacent to the plurality of mixing plates and configured to direct the combined substance to a distal end of the mixer attachment, wherein the mixer comprises a rod comprising one or more channels configured to facilitate mixing of the combined substance as it passes to the distal end of the mixer attachment.

19. The surgical hand piece of claim 18, wherein the one or more channels of the mixer are configured to facilitate diverging and converging flow of the combined substance during mixing.

20. The surgical hand piece of claim 11, further comprising a dose stop mechanism.
